(19) United States
(12) Patent Application Publication
(10) Pub. No.: US 2007/0259933 A1
(43) Pub. Date: Nov. 8, 2007

(54) COMPOSITIONS, DOSAGE FORMS AND METHODS OF TREATING EMESIS

(75) Inventors: Peter VIRSIK, Portola Valley, CA (US); Feng Xu, Palo Alto, CA (US)

Correspondence Address: Timothy A. Worrall, Ph.D., J.D.;Dorsey & Whitney LLP, 3rd Floor, 555 California Street, San Francisco, CA, 94104-1513, US

(73) Assignee: XenoPort, Inc.

(21) Appl. No.: 11/743,624

(22) Filed: May 2, 2007

Related U.S. Application Data
(60) Provisional application No. 60/798,106, filed on May 4, 2006.

(51) Int. Cl. A61K 31/4184; A61K 31/41; A61K 31/265

(52) U.S. Cl. 514/381; 514/394; 514/512

(57) ABSTRACT

Pharmaceutical compositions comprising an anti-emetic compound and a highly orally bioavailable form of propofol, oral dosage forms comprising an anti-emetic compound and a highly orally bioavailable form of propofol and methods of treating emesis in a patient comprising orally administering a therapeutically effective amount of an anti-emetic compound and a highly orally bioavailable form of propofol are disclosed.

This application claims the benefit of U.S. Provisional Application No. 60/798,106 filed May 4, 2006, which is incorporated by reference herein in its entirety.

### FIELD

Disclosed herein are pharmaceutical compositions for treating emesis comprising an anti-emetic compound and a highly orally bioavailable form of propofol, oral dosage forms comprising an anti-emetic compound and a highly orally bioavailable form of propofol, and methods of treating emesis in a patient comprising orally administering a therapeutically effective amount of an anti-emetic compound and a highly orally bioavailable form of propofol.

### BACKGROUND

Nausea, vomiting, and retching are basic human protective reflexes against the absorption of toxins as well as responses to certain stimuli. Nausea is a subjectively unpleasant wavelike sensation in the back of the throat or epigastrium associated with pallor or flushing, tachycardia, and an awareness of the urge to vomit. Sweating, excess salivation, and a sensation of being cold or hot may also occur. Vomiting is characterized by contraction of the abdominal muscles, descent of the diaphragm, and opening of the gastric cardia, resulting in forceful expulsion of stomach contents from the mouth. Retching involves spasmodic contractions of the diaphragm and the muscles of the thorax and abdominal wall without expulsion of gastric contents. Emesis is used herein to refer to nausea, vomiting, and/or retching.

The activation of a nucleus of neurons located in the medulla oblongata, known as the vomiting center, initiates the vomiting reflex. The vomiting center can be activated directly by signals from the cerebral cortex (anticipation, fear, memory), by signals from sensory organs (disturbing sights, smells, pain), or by signals from the vestibular apparatus of the inner ear (motion sickness). The vomiting center can also be activated indirectly by certain stimuli that activate the chemoreceptor trigger zone (CTZ). The CTZ is located in the highly vascular area postrema on the surface of the brain. Because this area lacks a true blood-brain barrier and is exposed to both blood and cerebrospinal fluid, the CTZ can react directly to substances in the blood. The CTZ can also be activated by signals from the stomach and small intestine traveling along vagal afferent nerves or by the direct action of emetogenic compounds that are carried in the blood such as, for example, chemotherapy drugs or opioids.

Specific neurotransmitters and neuromodulators in the CTZ identify substances as potentially harmful and relay impulses to the vomiting center to initiate the vomiting cascade so that the harmful substances can be expelled. These neurotransmitters include serotonin, dopamine, acetylcholine (muscarinic cholinergic), histamine, and neurokinin-1 (NK-1) neuropeptide. Stimulation of these chemoreceptors triggers activation of the vomiting center. Therefore, any interference with the transmission of these chemoreceptors can prevent the vomiting center from being activated. Many anti-emetics act by blocking one or more of these receptors. For example, dopamine antagonists block dopamine receptors; muscarinic antagonists block acetylcholine receptors; histamine blockers block histamine receptors; serotonin receptor blockers block serotonin receptors; and NK-1 receptor antagonists block NK-1 receptors. The adverse effects of these drugs are also determined by which receptor site is blocked.

Chemotherapy-induced nausea and vomiting (CINV) and post-operative nausea and vomiting (PONV) are two of the most significant targets of anti-emetic therapy. Chemotherapeutic agents used, for example, in cancer therapy can stimulate enterochromaffin cells in the gastrointestinal tract to release serotonin, which activates serotonin receptors. Activation of serotonin receptors subsequently activates the vagal afferent pathway, which in turn activates the vomiting center and causes an emetic response. The emetic potential of a chemotherapeutic agent can be the major stimulus for emesis in chemotherapy-induced emesis. Chemotherapeutic agents are rated according to their emetic potential.

CINV exhibits several characteristic temporal patterns. Anticipatory emesis occurs before the beginning of a new cycle of chemotherapy in response to conditioned stimuli such as the smells, sights, and sounds of the treatment room or the presence of a specific person who administers the chemotherapy. Anticipatory emesis usually occurs about 12 hours before administration of chemotherapy in patients who have experienced failed control of emesis. Acute emesis occurs within the first about 24 hours after the administration of chemotherapy. Delayed emesis begins at least 24 hours after initiation of chemotherapy and can last up to about 120 hours. The causative mechanism in delayed emesis is not well defined, however metabolites of an administered chemotherapeutic agent are thought to continue to affect the central nervous system and the gastrointestinal tract. For example, cisplatin causes delayed emesis up to about 48 to about 72 hours after administration in more than half of all patients who receive the drug. Breakthrough emesis occurs despite preventive therapy and requires additional anti-emetic treatment.

Due to the different etiologies, the different types of CINV are most effectively treated using different classes of anti-emetic agents. Chemotherapeutic agents initiate activation mainly of serotonin receptors (5-HT), which leads to the emetic response and therefore serotonin receptor antagonists are clinically effective drugs for treating acute CINV. Because serotonin receptor antagonists prevent emesis by blocking the emetic response early in the emetic pathway, the drugs can be given to patients before chemotherapy to prevent CINV. Examples of clinically useful serotonin receptor antagonists include ondansetron (Zofran®), granisetron (Kytril®), dolasetron (Anzemet®), and palonosetron (Aloxi®). Because serotonin receptor antagonists are less effective in treating anticipatory, delayed, and breakthrough CINV, the drugs can also be used in combination with other anti-emetic agents to provide more comprehensive anti-emetic therapy. For example, anticipatory emesis can be treated using limbic system inhibitors such as lorazepam (Ativan®), delayed CINV using corticosteroids such as dexamethasone or methylprednisolone (Solu-Medrol®), and breakthrough CINV using dopamine receptor antagonists such as prochlorperazine (Compazine®), metoclopramide (Reglan®), haloperidol (Haldol®), or dronabinol (Marinol®).

The need for anti-emetic agents and therapies that address both acute and delayed emesis associated with cancer chemotherapy is highlighted by the approvals of Aloxi® and Emend®. Palonsetron (Aloxi®) is a 5-HT₃ receptor antagonist with a long half-life and is the only 5-HT₃ antagonist that is approved in the U.S. for the prevention of both acute and delayed emesis. Aprepitant (Emend®) is a NK-1 receptor antagonist that belongs to a new class of anti-emetic compounds and is approved for the treatment of severe and moderate CINV. Guidelines for the prevention of CINV in patients undergoing highly emetogenic chemotherapy recommend the use of aprepitant in combination with a 5-HT₃ receptor antagonist and dexamethasone. While such drugs can be effective for treating acute and delayed emesis, a significant number of patients still experience emesis, indicating the need for improved anti-emetic compounds and treatments.

Post-operative nausea and vomiting (PONV) occurs after receiving anesthesia, such as during surgery. In post-operative nausea and vomiting a wide range of stimuli contribute to the emetic response. Most anesthetic agents and opioids stimulate the vomiting center indirectly through the CTZ. Associated factors that directly stimulate the vomiting center in PONV include sensory inputs, including visual, olfactory, and pain, and the vestibular apparatus. Other anesthetics such as nitrous oxide directly stimulate the gastrointestinal tract, which activates the vomiting center. Several classes of compounds including serotonin 5-HT₃ receptor antagonists have been shown safe and effective for the management of PONV (Gan, CNS Drugs 2005, 19(3), 225-238).

Because the etiology of nausea and vomiting is multifactorial, it has been suggested that combination anti-emetic therapy using a combination of agents acting at different receptor sites and/or a multimodal approach in which, in addition to administering anti-emetic agents that directly interfere with emetic response pathways, stimuli associated with the risk of developing emesis be minimized (see, Habib and Gan, Can. J. Anesth 2004, 51(4), 326-341).

In a number of studies, the administration of propofol has been shown effective in treating emesis. Propofol (2,6-diisopropylphenol), (1),

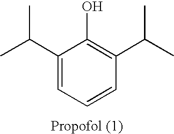

is a low molecular weight phenol that is widely used as an intravenous sedative-hypnotic agent in the induction and maintenance of anesthesia and/or sedation in mammals. The advantages of propofol as an anesthetic include rapid onset of anesthesia, rapid clearance, and minimal side effects (Langley et al., Drugs 1988, 35, 334-372). The hypnotic effects of propofol may be mediated through interaction with the GABA_{A} receptor complex, a hetero-oligomeric ligand-gated chloride ion channel (Peduto et al., Anesthesiology 1991, 75, 1000-1009).

Propofol also has a broad range of other biological and medical applications, which are evident at sub-anesthetic (e.g., sub-hypnotic) and sub-sedative doses. When used to maintain anesthesia, propofol causes a lower incidence of PONV when compared to common inhalation anesthetic agents and numerous controlled clinical studies support the anti-emetic activity of propofol (Tramer et al., Br. J. Anaesth. 1997, 78, 247-255; Brooker et al., Anaesth. Intensive Care 1998, 26, 625-629; Gan et al., Anesthesiology 1997, 87, 779-784; and Rudra et al., Indian J. Anaesth. 2004, 48(1), 31-34, each of which is incorporated by reference herein in its entirety). Sub-hypnotic doses of propofol administered post-operatively have also been shown effective in reducing PONV (see, Gan et al., Anesthesiology, 1999, 90, 1564-70; Gan et al. Anesthesiology 1996, 85, 1036-42; Borgeat et al., Anesth Analg 1992, 74, 539-541; Sculman et al., Anesth Analg 1995, 80, 636-637; Kim et al., Br. J. Anaesth. 2000, 85, 898-900; and Gan et al. Anaesthesiology 1997, 87, 779-84, each of which is incorporated by reference herein in its entirety). Propofol has also been shown to have anti-emetic activity for treating delayed emesis associated with CINV when used in conjunction with chemotherapeutic compounds (see, Phelps et al., Ann. Pharmacother 1996, 30, 290-292; Borgeat et al., Oncology 1993, 50, 456-459; Borgeat et al., Can. J. Anaesth. 1994, 41(11), 1117-1119; Tomioka et al., Anesth. Analg. 1999, 89, 798-799); and Scher et al., Can J. Anaesth 1992, 39(2), 170-172, each of which is incorporated by reference herein in its entirety). Emesis induced by a variety of chemotherapeutic agents such as cisplatin, cyclophosphamide, 5-fluorouracil, methotrexate, anthracycline drugs, etc., has been controlled by low-dose propofol infusion in patients refractory to prophylaxis with conventional anti-emetic drugs, such as serotonin antagonists and corticosteroids. For example, continuous propofol infusion at sub-hypnotic levels and the use of patient-controlled anti-emesis with propofol were found to be effective in the treatment of PONV (Kim et al., Br. J. Anaesth 2000, 85, 898-900; and Gan et al., Anesthesiology 1999, 90, 1564-70, each of which is incorporated by reference herein in its entirety).

The mechanism by which propofol prevents emesis is not known. Propofol may have a direct depressant effect on the chemoreceptor trigger zone, the vagal nuclei, and/or other centers implicated in the emetic response. It is postulated that the anti-emetic effects of propofol may be mediated through antagonism of the 5-HT₃ receptor (Hammas et al. Acta Anaesthesiol Scand 1998, 42, 447-51) or due to modulation of subcortical pathways (Borgeat et al., Oncology 1993, 50, 456-459). It is also proposed that propofol may act via an anti-dopaminergic pathway (Difloio, Anesth Analg 1993, 77, 200-201). For example, it has been reported that prolonged propofol infusion causes a decreased concentration of serotonin in the area postrema (Diab and Gelb, Neuroscience 1994, 20, 1169) and that this may be mediated through a GABA_{A} receptor mechanism (Gelb and Diab, Anesthesiology 1995, 83, A752). Propofol has also been shown to decrease synaptic transmission in the olfactory cortex suggesting a decrease in the release of excitatory amino acids such as glutamate and aspartate, which may be related to its anti-emetic activity.

Continuous infusion of propofol at a low sub-hypnotic dose of about 1 mg/kg/h (about 17 μg/kg/min) in conjunction with the 5-HT₃ receptor antagonist, ondansetron, and the corticosteroid, dexamethasone, has been shown to reduce emesis in patients receiving cisplatin chemotherapy (Borgeat et al., Oncology 1993, 50, 456-459, which is incorporated by reference herein in its entirety). A continuous intravenous dose of about 1 mg/kg/h is much less than the dose necessary to maintain anesthesia, e.g. about 8 mg/kg/h to about 12 mg/kg/h (Id.), and corresponds to a propofol plasma concentration from about 400 ng/mL to about 540 ng/mL (Gan et al., Anesthesiology 1997, 87(4), 779-784, which is incorporated by reference herein in its entirety). In a study evaluating the use of propofol to treat PONV, Gan et al. determined that plasma concentrations of propofol from about 200 ng/mL to about 600 ng/mL (10%-90% percentile) during a 24-hour period were effective in lowering the incidence of PONV (Gan et al., Anesthesiology 1997, 87(4), 779-784). Again, these ranges are much lower than the propofol plasma concentrations needed for sedation (about 1,500-2,000 ng/mL) and for the maintenance of general anesthesia (about 3,000-10,000 ng/mL).

Propofol is rapidly metabolized in mammals with the drug being eliminated predominantly as glucuronidated and sulfated conjugates of propofol and 4-hydroxypropofol (Langley et al., Drugs 1988, 35, 334-372). Propofol is poorly absorbed in the gastrointestinal tract and only from the small intestine. When orally administered as a homogeneous liquid suspension, propofol exhibits an oral bioavailability of less than 5% that of an equivalent intravenous dose of propofol. Propofol clearance exceeds liver blood flow, which indicates that extrahepatic tissues contribute to the overall metabolism of the drug. Human intestinal mucosa glucuronidates propofol in vitro and oral dosing studies in rats indicate that approximately 90% of the administered drug undergoes first pass metabolism, with extraction by the intestinal mucosa accounting for the bulk of this pre-systemic elimination (Raoof et al., Pharm. Res. 1996, 13, 891-895). Because of its poor bioavailability and extensive first-pass metabolism, propofol is administered by injection or intravenous infusion and oral administration of propofol has not been considered therapeutically effective. Recently, several methods for improving propofol absorption from the gastrointestinal tract and/or minimizing first pass metabolism have been demonstrated.

Propofol prodrugs that exhibit enhanced oral bioavailability and that are sufficiently labile under physiological conditions to provide therapeutically effective concentrations of propofol have been described Gallop et al., U.S. Application Publication No. 2005/0004381; Gallop et al., U.S. Application Publication No. 2005/0107385; Xu et al., U.S. Application Publication No. 12006/0041011; Xu et al., U.S. Application Publication No. 2006/0100160; and Xu et al., U.S. Application Publication No. 2005/0265609, each of which is incorporated by reference herein in its entirety. In view of the demonstrated efficacy of propofol in preventing both acute and delayed emesis, such propofol prodrugs, when orally administered, optionally in combination with other anti-emetic compounds such as, for example, 5-HT₃ receptor antagonists and/or corticosteroids, can be useful in treating emesis, and in particular, CINV and PONV.

The ability to provide therapeutically effective plasma and/or blood concentrations of propofol via an oral dosage form, optionally in combination with one or more additional anti-emetic compounds can improve therapeutic efficacy and facilitate anti-emetic therapy post-discharge.

Accordingly, in a first aspect, pharmaceutical compositions are provided comprising a first anti-emetic compound selected from a serotonin 5-HT₃ receptor antagonist, a histamine receptor antagonist, a dopamine receptor antagonist, a muscarinic receptor antagonist, an acetylcholine receptor antagonist, a cannabinoid receptor antagonist, a limbic system inhibitor, a NK-1 receptor antagonist, a corticosteroid, a tachykinin antagonist, a GABA agonist, a substance P inhibitor, and combinations of any of the foregoing, and a highly orally bioavailable form of propofol that exhibits an oral bioavailability that is at least 10 times greater than the oral bioavailability of propofol when orally administered in an equivalent dosage form.

In a second aspect, oral dosage forms for treating emesis in a patient are provided comprising a first anti-emetic compound selected from a serotonin 5-HT₃ receptor antagonist, a histamine receptor antagonist, a dopamine receptor antagonist, a muscarinic receptor antagonist, an acetylcholine receptor antagonist, a cannabinoid receptor antagonist, a limbic system inhibitor, a NK-1 receptor antagonist, a corticosteroid, a tachykinin antagonist, a GABA agonist, a substance P inhibitor, and combinations of any of the foregoing, and a highly orally bioavailable form of propofol, wherein the oral dosage form is adapted to provide, after a single oral administration of the oral dosage form to the patient, a therapeutically effective concentration of the first anti-emetic compound in the plasma of the patient during a continuous time period selected from at least about 4 hours, at least about 8 hours, at least about 12 hours, and at least about 16 hours, and at least about 20 hours, and a therapeutically effective concentration of propofol in the plasma of the patient during a continuous time period independently selected from at least about 4 hours, at least about 8 hours, at least about 12 hours, at least about 16 hours, and at least about 20 hours.

In a third aspect, methods of treating emesis in a patient comprising administering to a patient in need of such treatment a therapeutically effective amount of a pharmaceutical composition provided by the present disclosure.

In a fourth aspect, methods of treating emesis in a patient comprising administering to a patient in need of such treatment a therapeutically effective amount of an oral dosage form provided by the present disclosure.

In a fifth aspect, methods of treating emesis in a patient are provided comprising administering to a patient in need of such treatment a therapeutically effective amount of a first anti-emetic compound selected from a serotonin 5-HT₃ receptor antagonist, a histamine receptor antagonist, a dopamine receptor antagonist, a muscarinic receptor antagonist, an acetylcholine receptor antagonist, a cannabinoid receptor antagonist, a limbic system inhibitor, a NK-1 receptor antagonist, a corticosteroid, a tachykinin antagonist, a GABA agonist, a substance P inhibitor, and combinations of any of the foregoing, and an oral dosage form comprising a highly orally bioavailable form of propofol, wherein the oral dosage from is adapted to provide, after a single oral administration of the oral dosage form to the patient a therapeutically effective concentration of propofol in the plasma of the patient during a continuous time period independently selected from at least about 4 hours, at least about 8 hours, at least about 12 hours, at least about 16 hours, and at least about 20 hours.

### DETAILED DESCRIPTION

### Definitions

A dash (“—”) that is not between two letters or symbols is used to indicate a point of attachment for a moiety or substituent. For example, —CONH₂ is attached through the carbon atom.

“Alkyl” by itself or as part of another substituent refers to a saturated or unsaturated, branched or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl),; prop-1-yn-1-yl, prop-2-yn-1-yl, etc.; butyls such as butan-1-yl, butan-2-yl, 2-methyl-propan-1-yl, 2-methyl-propan-2-yl, , but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the expressions “alkanyl,” “alkenyl,” and “alkynyl” are used. In certain embodiments, an alkyl group comprises from 1 to 20 carbon atoms, in certain embodiments, from 1 to 10 carbon atoms, from 1 to 6 carbon atoms, and in certain embodiments, from 1 to 3 carbon atoms.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, or heteroarylalkyl as defined herein. Examples of acyl groups include, but are not limited to, formyl, acetyl, cyclohexylcarbonyl, cyclohexylmethylcarbonyl, benzoyl, benzylcarbonyl, and the like.

“Alkoxy” by itself or as part of another substituent refers to a radical —OR³ where R³¹ is chosen from alkyl, heteroalkyl, cycloalkyl, heterocycloalkyl, cycloalkylalkyl, cycloheteroalkylalkyl, aryl, heteroaryl, arylalkyl, and heteroarylalkyl, as defined herein. Examples of alkoxy groups include, but are not limited to, methoxy, ethoxy, propoxy, butoxy, cyclohexyloxy, and the like.

“Alkoxycarbonyl” by itself or as part of another substituent refers to a radical —C(O)OR³² where R³² represents an alkyl or cycloalkyl group as defined herein. Examples of alkoxycarbonyl groups include, but are not limited to, methoxycarbonyl, ethoxycarbonyl, propoxycarbonyl, butoxycarbonyl, cyclohexyloxycarbonyl, and the like.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocylic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, s-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms, from 6 to 12 carbon atoms, and in certain embodiments, from 6 to 8 carbon atoms. Aryl does not encompass or overlap in any way with heteroaryl, separately defined herein. Hence, a multiple ring system in which one or more carbocyclic aromatic rings is fused to a heterocycloalkyl aromatic ring, is heteroaryl, not aryl, as defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, and/or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“AUC” is the area under a curve representing the concentration of a compound in a biological fluid in a patient as a function of time following administration of the compound to the patient. In certain embodiments, the compound can be a prodrug and the metabolite can be a drug. Examples of biological fluids include plasma and blood. The AUC can be determined by measuring the concentration of a compound in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. As relevant to the present disclosure, an AUC for propofol can be determined by measuring the concentration of propofol in the plasma or blood of a patient following oral administration of a dosage form comprising a form of propofol, such as a propofol prodrug or a propofol tight-ion pair complex.

“Bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under the curve (AUC), the time to peak concentration (Tₘₐₓ), and the maximum drug concentration (Cₘₐₓ), where Cₘₐₓ is the maximum concentration of a drug in the plasma or blood of a patient following administration of a dose of the drug or form of drug to the patient, and Tₘₐₓ is the time to the maximum concentration (Cₘₐₓ) of a drug in the plasma or blood of a patient following administration of a dose of the drug or form of drug to the patient.

“Cₘₐₓ” is the maximum concentration of a drug in the plasma or blood of a patient following administration of a dose of the drug or prodrug to the patient.

“Tₘₐₓ” is the time to the maximum (peak) concentration (Cₘₐₓ) of a drug in the plasma or blood of a patient following administration of a dose of the drug or prodrug to the patient.

“Carbamoyl” by itself or as part of another substituent refers to the radical —C(O)NR³⁹R⁴⁰ where R³⁹ and R⁴⁰ are independently hydrogen, alkyl, cycloalkyl, or aryl as defined herein.

“Carboxyl” refers to the group —COOH.

“Compounds” refers to compounds encompassed by any of the structural formulae disclosed herein and includes any specific compounds within these formulae whose structure is disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers, or diastereomers. Accordingly, the chemical structures depicted herein encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form, and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds disclosed herein include, but are not limited to ²H, ³H, ¹¹C, ¹³C, ¹⁴C, ¹⁵N, ¹⁸O, ¹⁷O etc. Compounds may exist in unsolvated forms as well as solvated forms, including hydrated forms and as N-oxides. In general, compounds may be hydrated, solvated, or N-oxides. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

Further, when partial structures of the compounds are illustrated, an asterisk (*) indicates the point of attachment of the partial structure to the rest of the molecule.

“Cycloalkoxycarbonyl” by itself or as part of another substituent refers to a radical —C(O)OR³⁶ where R³⁶ represents an cycloalkyl group as defined herein. Examples of cycloalkoxycarbonyl groups include, but are not limited to, cyclobutyloxycarbonyl, cyclohexyloxycarbonyl, and the like.

“Cycloalkyl” by itself or as part of another substituent refers to a saturated or partially unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₀ cycloalkyl, and in certain embodiments, C₃₋₇ cycloalkyl.

“Cycloheteroalkyl” by itself or as part of another substituent refers to a saturated or partially unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Examples of cycloheteroalkyl groups include, but are not limited to, groups derived from epoxides, azirines, thiiranes, imidazolidine, morpholine, piperazine, piperidine, pyrazolidine, pyrrolidine, quinuclidine, and the like.

“Dosage form” means a pharmaceutical composition in a medium, carrier, vehicle, or device suitable for administration to a patient.

“Emesis” as used herein, means nausea, vomiting, and/or retching, either independently or in combination. An emetic response refers to nausea, vomiting, and/or retching. Nausea is a subjectively unpleasant wavelike sensation in the back of the throat or epigastrium associated with pallor or flushing, tachycardia, and an awareness of the urge to vomit. Sweating, excess salivation, and a sensation of being cold or hot may also occur. Vomiting is characterized by contraction of the abdominal muscles, descent of the diaphragm, and opening of the gastric cardia, resulting in forceful expulsion of stomach contents from the mouth. Retching involves spasmodic contractions of the diaphragm and the muscles of the thorax and abdominal wall without expulsion of gastric contents.

“Halogen” refers to a fluoro, chloro, bromo, or iodo group.

“Heteroalkyl” by itself or as part of another substituent refer to an alkyl group in which one or more of the carbon atoms (and any associated hydrogen atoms) are independently replaced with the same or different heteroatomic groups. Examples of heteroatomic groups include, but are not limited to, —O—, —S—, —O—O—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —SO—, —SO₂—, —SnR⁴³R⁴⁴—, and the like, where R³⁷, R³⁸, R³⁹, R⁴⁰, R⁴¹, R⁴², R⁴³, and R⁴⁴ are independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ cycloheteroalkyl, substituted C₃₋₇ cycloheteroalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. Where a specific level of saturation is intended, the nomenclature “heteroalkanyl,” “heteroalkenyl,” or “heteroalkynyl” is used. In certain embodiments, R³⁷, R³⁸, R³⁹, R⁴⁰, R⁴¹, R⁴², R⁴³, and R⁴⁴ are independently chosen from hydrogen and C₁₋₃ alkyl.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. Heteroaryl encompasses 5- to 7-membered aromatic, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heteroaromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, in certain embodiments from 5- to 10-membered heteroaryl, and in certain embodiments from 6- to 8-heteroaryl. In certain embodiments heteroaryl groups are those derived from thiophene, pyrrole, benzothiophene, benzofuran, indole, pyridine, quinoline, imidazole, oxazole, or pyrazine.

“Heteroarylalkyl” by itself or as part of another substituent refers to an acyclic alkyl radical in which one of the hydrogen atoms bonded to a carbon atom, is replaced with a heteroaryl group. Typically a terminal or sp³ carbon atom is the atom replaced with the heteroaryl group. Where specific alkyl moieties are intended, the nomenclature “heteroarylalkanyl,” “heteroarylalkenyl,” and “heterorylalkynyl” is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl

“Highly orally bioavailable form of propofol” means a form of propofol that when orally administered provides an oral bioavailability of propofol that is at least about 10%, in certain embodiments at least about 20%, and in certain embodiments, at least about 40%, of the propofol bioavailability following intravenous administration of an equivalent dose of propofol. A “highly orally bioavailable form of propofol” also means a form of propofol that exhibits an oral bioavailability of propofol that is at least about 5 times greater, in certain embodiments at least about 10 time greater, and in certain embodiments at least about 20 times greater than the oral bioavailability of propofol when an equivalent amount of propofol is orally administered to a patient in an equivalent dosage form. A “highly orally bioavailable form of propofol” also means a form of propofol that exhibits an oral bioavailability of propofol that is at least about 5 times greater, in certain embodiments at least about 10 time greater, and in certain embodiments at least about 20 times greater, than the oral bioavailability of propofol when an equivalent amount of propofol is orally administered to a patient as a uniform liquid immediate release formulation.

The absolute oral bioavailability of orally administered propofol is less than about 2%. The absolute oral bioavailability is the dose-normalized bioavailability of orally administered propofol divided by the dose-normalized bioavailability of intravenously administered propofol, times 100.

Oral bioavailability can be determined, for example by administering solutions or suspensions containing a highly bioavailable form of propofol or propofol to animals, e.g., rats and dogs, via oral gavage.

“Hydroxyl” refers to the group —OH.

“Parent aromatic ring system” refers to an unsaturated cyclic or polycyclic ring system having a conjugated π electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, and the like. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” refers to a mammal, for example, a human.

“Pharmaceutical composition” refers to a composition comprising a compound effective for treating a disease, disorder, or condition, and at least one pharmaceutically acceptable vehicle with which the compound is administered to a patient.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound

“Prodrug of propofol” refers to a compound in which a promoiety, which is cleavable in vivo, is covalently bound to the propofol molecule. In certain embodiments, the prodrug can be actively transported by transporters expressed in the enterocytes lining the gastrointestinal tract such as, for example, the PEPT1 transporter. Propofol prodrugs provided by the present disclosure are stable in the gastrointestinal tract and following absorption are cleaved in the systemic circulation to release propofol. In certain embodiments, a prodrug of propofol provides a greater oral bioavailability of propofol compared to the oral bioavailability of propofol when administered as a uniform liquid immediate release formulation. In certain embodiments, a prodrug of propofol is highly orally bioavailable, exhibiting an oral bioavailability that is at least 10 times greater than the oral bioavailability of propofol when orally administered in an equivalent dosage form. In certain embodiments, a prodrug of propofol is a compound having a structure encompassed by any one of Formulae (I)-(XIII), infra.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a patient to which the prodrug is administered or the acidic conditions of the stomach, or the agent may be supplied exogenously. For example, for a prodrug of Formula (XI), the drug is propofol and the promoiety has the structure:

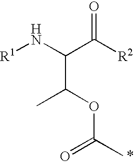

where R¹ and R² are defined herein.

“Solvate” refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules are water.

“Controlled delivery” means continuous or discontinuous release of a drug over a prolonged period of time, wherein the drug is released at a controlled rate over a controlled period of time in a manner that provides for upper gastrointestinal and lower gastrointestinal tract delivery, coupled with improved drug absorption as compared to the absorption of the drug in an immediate release oral dosage form.

“Sustained release” refers to release of a therapeutic amount of a drug, a prodrug, or an active metabolite of a prodrug over a period of time that is longer than that of a conventional formulation of the drug, e.g. an immediate release formulation of the drug. For oral formulations, the term “sustained release” typically means release of the drug within the gastrointestinal tract lumen over a time period from about 2 to about 30 hours, and in certain embodiments, over a time period from about 4 to about 24 hours. Sustained release formulations achieve therapeutically effective concentrations of the drug in the systemic circulation over a prolonged period of time relative to that achieved by oral administration of a conventional formulation of the drug. “Delayed release” refers to release of a drug, a prodrug, or an active metabolite of a prodrug into the gastrointestinal lumen after a delayed time period, for example a delay of about 1 to about 12 hours, relative to that achieved by oral administration of a conventional formulation of the drug.

“Substantially one diastereomer” refers to a compound containing 2 or more stereogenic centers such that the diastereomeric excess (d.e.) of the compound is greater than or at least about 90%. In certain embodiments, the d.e. is, for example, greater than or at least about 91%, about 92%, about 93%, about 94%, about 95%, about 96%, about 97%, about 98%, or about 99%.

“Substituted” refers to a group in which one or more hydrogen atoms are independently replaced with the same or different substituent(s). Typical substituents include, but are not limited to, -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is independently a halogen; R⁶⁰, R⁶¹, R⁶², and R⁶³ are independently selected from hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl, and substituted heteroaryl, or R⁶⁰ and R⁶¹ together with the nitrogen atom to which they are bonded form a cycloheteroalkyl or substituted cycloheteroalkyl ring. In certain embodiments, R⁶⁰, R⁶¹, R⁶², and R⁶³ are independently selected from hydrogen, C₁₋₆ alkyl, C₁₋₆ alkoxy, C₃₋₁₂ cycloalkyl, C₃₋₁₂ cycloheteroalkyl, C₆₋₁₂ aryl, and C₆₋₁₂ heteroaryl. In certain embodiments, each substituent group is independently selected from halogen, —OH, —CN, —CF₃, ═O, —NO₂, C₁₋₃ alkoxy, C₁₋₃ alkyl, —COOR⁶⁴ wherein R⁶⁴ is selected from hydrogen and C₁₋₃ alkyl, and —NR⁶⁵₂ wherein each R⁶⁵ is independently chosen from hydrogen and C₁₋₃ alkyl. In certain embodiments, each substituent group is independently selected from halogen, —OH, C₁₋₃ alkyl, and C₁₋₃ alkoxy.

“Therapeutically effective amount” refers to the amount of a compound that, when administered to a subject for treating a disease or disorder, or at least one of the clinical symptoms of a disease or disorder, is sufficient to affect such treatment of the disease, disorder, or symptom. The “therapeutically effective amount” may vary depending, for example, on the compound, the disease, disorder, and/or symptoms of the disease or disorder, severity of the disease, disorder, and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or disorder or at least one or more symptoms thereof in a patient which may be exposed to or predisposed to a disease or disorder even though that patient does not yet experience or display symptoms of the disease or disorder.

As used herein, treating emesis includes preventing sensations of emesis, preventing episodes of emesis from occurring, reducing the severity of emesis experienced by the patient, minimizing the number of episodes of emesis, and/or minimizing the frequency of episodes of emesis.

“Upper gastrointestinal tract” means that portion of the gastrointestinal tract including the stomach and the small intestine.

“Intestine” or “gastrointestinal tract” means the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

“Lower gastrointestinal tract” means the large intestine.

“Window” means a period of time having defined duration. Windows can begin at the time of administration of a dosage form to a patient, or any time thereafter. For example, in certain embodiments a window can have a duration of about 12 hours. In certain embodiments, a window can begin at a variety of times. For example, in certain embodiments, a window can begin about 1 hour after administration of a dosage form, and have a duration of about 12 hours, which means that the window would open about 1 hour after administration of the dosage form and close at about 13 hours following administration of the dosage form.

Reference is now be made in detail to certain embodiments of pharmaceutical compositions, dosage forms, and methods. The disclosed embodiments are not intended to be limiting of the claims. To the contrary, the claims are intended to cover all alternatives, modifications, and equivalents.

### Pharmaceutical Compositions

Pharmaceutical compositions provided by the present disclosure comprise an anti-emetic compound selected from a serotonin 5-HT₃ receptor antagonist, a histamine receptor antagonist, a dopamine receptor antagonist, a muscarinic receptor antagonist, an acetylcholine receptor antagonist, a cannabinoid receptor antagonist, a limbic system inhibitor, a NK-1 receptor antagonist, a corticosteroid, a tachykinin antagonist, a GABA agonist, a substance P inhibitor, and combinations of any of the foregoing, and a highly orally bioavailable form of propofol that exhibits an oral bioavailability that is at least 10 times greater than the oral bioavailability of propofol when administered in an equivalent dosage form.

An anti-emetic compound is a compound that reduces the likelihood of emesis, prevents emesis from occurring, reduces the severity of emesis, and/or minimizes the number of or frequency of episodes of emesis.

As discussed, supra, many anti-emetics block one or more of the chemoreceptors involved in the emetic response. These include dopamine receptor antagonists such as promethazine; phenothiazines such as chlorpromazine and prochlorperazine; butyrophenones such as droperidol and haloperidol; and benzamides such as metocloproamide, cisapride, and trimethobenzamide; histamine receptor antagonists include antihistamines such as dimenhydrinate, meclizine, diphenhydramine, cyclizine, and promethazine; muscarininic cholinergic receptor antagonists include anticholinergics such as scopolamine and hyoscine; neurokinin-1 receptors (NK-1) antagonists include aprepitant; and serotonin receptor antagonists including 5-HT₃ receptor antagonists.

Serotonin 5-HT₃ receptor antagonists are widely used in the management of CINV and PONV. Serotonin, also referred to as 5-hydroxytryptamine (5-HT), acts both centrally and peripherally on discrete 5-HT receptors. Currently, fourteen subtypes of serotonin receptors are recognized and delineated into seven families, 5-HT through 5-HT₇ (see Martin and Humphrey, Neuropharm, 1994, 33(3-4), 261-273; Hoyer et al., Pharm. Rev., 1994, 46(2), 157-203). 5-HT₃ receptors are ligand-gated ion channels that are extensively distributed on enteric neurons in the human gastrointestinal tract, as well as other peripheral and central locations. Activation of these channels and the resulting neuronal depolarization has been found to affect the regulation of visceral pain, colonic transit, and gastrointestinal secretions. Antagonism of 5-HT₃ receptors also has the potential to influence sensory and motor function in the gut including the emetic response. 5-HT₃ receptor antagonists are believed to inhibit emesis by blocking vagal efferent nerve terminals in gastrointestinal mucosa and on terminals on the same vagal nerves in the vomiting system located in the dorsal medulla of the brain stem. 5-HT₃ receptor antagonists have been shown to be less effective for delayed emesis than for acute symptoms. In addition, the efficacy of the 5-HT₃ receptor antagonists appears to be less pronounced for moderate emetogenic chemotherapy regimens than for regimens employing chemotherapeutic agents having high emetogenic potential such as cisplatin cyclophospamide, doxorubicin, acarbazine, actinomycin D, mechlorethamine, treptozocin, hexamethylamine, lomustine, carmuistine, daunorubicin, epirubicin, idarubicin, oxaliplatin, cytarabine, and ifosfamide (see, e.g., National Comprehensive Cancer Network, “Clinical Practice Guidelines in Oncology,” v.1, 2006, for emetogenic potential of antineoplastic agents). Furthermore, control over nausea appears to be significantly less than control over vomiting and the efficacy of 5-HT₃ receptor antagonists appears to diminish over repeated days and across repeated chemotherapy cycles (see, e.g., Morrow et al., Cancer 1995, 76(3), 343-357).

Examples of 5-HT₃ receptor antagonists include indisetron, YM-114, granisetron, talipexole, azasetron, bemesetron, tropisetron, ramosetron, ondansetron, palonosetron, lerisetron, alosetron, N-3389, zacopride, cilansetron, E-3620, lintopride, KAE-393, itasetron, zatosetron, dolasetron, (±)-renzapride, (−)-YM-060, DAU-6236, BIMU-8, GK-128, Ro-93777, mirtazapine, mosapride, fabesetron, galdansetron, lurosetron, and ricasetron. In certain embodiments, a 5-HT₃ receptor antagonist useful in pharmaceutical compositions provided by the present disclosure is selected from alosetron, azasetron, bemesetron, cilansetron, dolasetron, granisetron, indisetron, itasetron, ondansetron, palonosetron, ramosetron, tropisetron, and zatosetron. In certain embodiments, a 5-HT₃ receptor antagonist useful in pharmaceutical compositions provided by the present disclosure is selected from alosetron, dolasetron, granisetron, ondansetron, and palonosetron. In certain embodiments, a serotonin 5-HT₃ receptor antagonist useful in pharmaceutical compositions provided by the present disclosure is ondansetron.

Pharmaceutical compositions provided by the present disclosure may include one serotonin 5-HT₃ receptor antagonist or more than one serotonin 5-HT₃ receptor antagonist.

Other anti-emetic compounds useful for treating emesis include corticosteroids such as dexamethasone and methylprednisolone; compounds that interact with cannabinoid receptor sites including cannabinoids such as dronabinol, tetrahydrocannabinol, and nabilone; limbic system inhibitors including benzodiazepines such as lorazepam, alprazolam, and midazolam; and tricyclic antidepressants (Prakash et al., Am. J. Gastroenterol 1999, 94(10), 2855-2860).

In certain embodiments, a pharmaceutical composition includes a serotonin 5-HT₃ receptor antagonist and a second anti-emetic compound selected from a histamine receptor antagonist, a dopamine receptor antagonist, a muscarinic receptor antagonist, an acetyl choline receptor antagonist, a cannabinoid receptor antagonist, a limbic system inhibitor, a NK-1 receptor antagonist, a corticosteroid, a tachykinin antagonist, a GABA agonist, a substance P inhibitor, and a combination of any of the foregoing. In certain embodiments, a second anti-emetic compound can be a corticosteroid selected from dexamethasone and methylprednisolone. In certain embodiments, a second anti-emetic compound is dexamethasone, and in certain embodiments, a second anti-emetic compound is methylprednisolone.

In certain embodiments, an anti-emetic compound can be useful for treating CINV. Examples of anti-emetic compounds useful for treating CINV include aprepitant, dexamethasone, dolasetron, dronabinol, granesetron, lorazepam, metoclopramide, ondansetron, and palonosetron.

In certain embodiments, an anti-emetic compound can be useful for treating PONV. Examples of anti-emetic compounds useful for treating PONV include dexamethasone, dolasetron, granisetron, metoclopramide, and ondansetron.

In certain embodiments, an anti-emetic compound can be useful for treating emesis induced by radiotherapy. Examples of anti-emetic compounds useful for treating emesis induced by radiotherapy include granisetron and ondansetron.

In certain embodiments, an anti-emetic compound can be useful for treating breakthrough emesis such as prochlorperazine, thiethylperazine, metoclopramide, diphenhydramine, lorzepam, haloperidol, or dronabinol.

In certain embodiments, a pharmaceutical composition can comprise a serotonin 5-HT₃ receptor antagonist and a corticosteroid. The 5-HT₃ receptor antagonist can be selected from alosetron, azasetron, bemesetron, cilansetron, dolasetron, granisetron, indisetron, itasetron, ondansetron, palonosetron, ramosetron, tropisetron, and zatosetron, and the corticosteroid can be selected from dexamethasone and methylprednisolone. In certain embodiments, a pharmaceutical composition can comprise ondansetron and dexamethasone.

Pharmaceutical compositions provided by the present disclosure comprise one or more highly orally bioavailable forms of propofol. In certain embodiments, a highly orally bioavailable form of propofol exhibits a propofol bioavailability following oral administration to a patient of at least about 10%, in certain embodiments at least about 20%, and in certain embodiments at least about 40%, of the propofol bioavailability following intravenous administration of an equivalent dose of propofol. In certain embodiments, a highly orally bioavailable form of propofol exhibits an oral bioavailability of propofol that is at least about 5 times greater, in certain embodiments at least about 10 time greater, and in certain embodiments at least about 20 times greater than the oral bioavailability of propofol when an equivalent amount of propofol is orally administered to a patient in an equivalent dosage form. In certain embodiments, a highly orally bioavailable form of propofol exhibits an oral bioavailability of propofol that is at least about 5 times greater, in certain embodiments at least about 10 time greater, and in certain embodiments at least about 20 times greater, than the oral bioavailability of propofol when an equivalent amount of propofol is orally administered to a patient as a uniform liquid immediate release formulation.

Highly orally bioavailable forms of propofol include prodrugs, conjugates, and complexes. A promoiety covalently (e.g., bonded) or non-covalently attached to propofol can enhance permeability through gastrointestinal epithelia via passive and/or active transport mechanisms, can control the release of propofol in the gastrointestinal tract, and/or can inhibit enzymatic and chemical degradation of propofol in the gastrointestinal tract. For highly orally bioavailable forms of propofol in which a promoiety remains bonded to the propofol molecule after absorption, the promoiety can enhance permeability through other biological membranes, and/or can inhibit enzymatic and chemical degradation of propofol in the systemic circulation.

Reducing the rate of metabolism of the drug in the gastrointestinal tract and/or enhancing the rate by which the drug is absorbed from the gastrointestinal tract can enhance the oral bioavailability of a drug. An orally administered drug will pass through the gastrointestinal system in about 11 to 31 hours. In general, an orally ingested drug resides about 1 to 6 hours in the stomach, about 2 to 7 hours in the small intestine, and about 8 to 18 hours in the colon. The oral bioavailability of a particular drug will depend on a number of factors including the residence time in a particular region of the gastrointestinal tract, the rate the drug is metabolized within the gastrointestinal tract, the rate the drug is metabolized in the systemic circulation, and the rate the drug or form of drug is absorbed from a particular region or regions of the gastrointestinal tract, which include passive and active transport mechanisms. Several methods have been developed to achieve these objectives, including drug modification, incorporating the drug or modified drug in a controlled release dosage form, and/or by co-administering adjuvants, which can be incorporated in the dosage form containing the active compound.

A drug can be modified to reduce the rate of drug metabolism in the gastrointestinal tract and/or to enhance and or modify the absorption of the drug from the gastrointestinal tract. Forms of propofol with enhanced oral bioavailability include propofol tight-ion pairs and propofol prodrugs.

Wong et al., U.S. Application Publication No. 2005/0163850 (which is incorporated by reference herein in its entirety) disclose forming tight-ion pair complexes of generally hydrophobic compounds such as alkyl sulfates or fatty acids. The tight-ion pair complexes disclosed by Wong et al. are characterized by a generally hydrophobic exterior and are intended to be more stable than loose ion pairs in the presence of water rendering the complexes more likely to move through intestinal epithelial membranes by paricellular or active transport. Such tight-ion pair complexes can enhance absorption of drugs as well as prodrugs in both the upper and lower gastrointestinal tract.

In certain embodiments, a form of propofol is a propofol prodrug. In certain embodiments, prodrugs of propofol can provide a greater oral bioavailability of propofol relative to the oral bioavailability of propofol when orally administered to a patient as a uniform liquid immediate release formulation and/or when orally administered in an equivalent dosage form. In certain embodiments, a prodrug of propofol can be a highly orally bioavailable form of propofol exhibiting an oral bioavailability that is at least 10 times greater than the oral bioavailability of propofol when orally administered in an equivalent dosage form. Examples of propofol prodrugs with enhanced oral bioavailability include bile acid prodrugs, peptide conjugates, and prodrugs in which propofol is bonded to an amino acid or small peptide via a linkage.

Prodrugs are compounds in which a promoiety is typically covalently bonded to a drug. Following absorption from the gastrointestinal tract, the promoiety is cleaved to release the drug into the systemic circulation. While in the gastrointestinal tract, the promoiety can protect the drug from the harsh chemical environment, and can also facilitate absorption. Promoieties can be designed, for example, to enhance passive absorption, e.g., lipophilic promoieties, and/or enhance absorption via active transport mechanisms, e.g., substrate promoieties. In particular, active transporters differentially expressed in regions of the gastrointestinal tract can be preferentially targeted to enhance absorption. For example, a propofol prodrug can incorporate a promoiety that is a substrate of PEPT1 transporters expressed in the small intestine. Zerangue et al., U.S. Application Publication Nos. 2003/0017964 and 2005/0214853 (each of which is incorporated by reference herein in its entirety) disclose methodologies for screening drugs, conjugates or conjugate moieties, linked or linkable to drugs, for their capacity to be transported as substrates via the PEPT1 and PEPT2 transporters, which are known to be expressed in the human small intestine (see, e.g., Fei et al., Nature 1964, 386, 563-566; and Miyamoto et al., Biochimica et Biophysica Acta 1996, 1305, 34-38). Zerangue et al., U.S. Application Publication No. 2003/0158254 also disclose several transporters expressed in the human colon including the sodium dependent multi-vitamin transporter (SMVT) and monocarboxylate transporters MCT1 and MCT4, methods of identifying agents or conjugate moieties that are transporter substrates, and agents, conjugates, and conjugate moieties that can be screened for substrate activity. Zerangue et al. further disclose compounds that can be screened that are variants of known transporter substrates such as bile salts or acids, steroids, ecosanoids, or natural toxins or analogs of any of the foregoing, as described by Smith, Am. J Physiol 1987, 223, 974-978; Smith, Am J Physio. 1993, 252, G479-G484; Boyer, Proc Natl Acad Sci USA 1993, 90, 435-438; Fricker, Biochem J 1994, 299, 665-670; Ficker, Biochem J 1994, 299, 665-670; and Ballatori et al., Am J Physiol 2000, 278 G57-G63, and the linkage of drugs to conjugate moieties.

Conjugation to bile acids has been shown to enhance oral bioavailability of a drug. Bile acids are hydroxylated steroids that play a key role in digestion and absorption of fat and lipophilic vitamins. After synthesis in the liver, bile acids are secreted into bile and excreted by the gall bladder into the intestinal lumen where they emulsify and help solubilize lipophilic substances. Bile acids are conserved in the body by active uptake from the terminal ileum via the sodium-dependent transporter IBAT (or ASBT) and subsequent hepatic extraction by the transporter NTCP (or LBAT) located in the sinusoidal membrane of hepatocytes. Gallop et al. disclose prodrugs in which a drug is covalently bonded to a cleavable linker which in turn is covalently bonded to a moiety, such as a bile acid or bile acid derivative, that facilitates translocation of the conjugate across the intestinal epithelia via the bile acid transport system (see, Gallop et al., U.S. Pat. Nos. 6,984,634, 6,900,192, and 6,984,634; and U.S. Application Publication Nos. 2002/0099041, 2005/0272710, 2003/0130246, 2005/0148564, and 2005/0288228, each of which is incorporated by reference herein in its entirety). Following absorption via the bile acid transport system, the linker is cleaved to release the drug into the systemic circulation.

Another drug-modification method includes covalent bonding drugs directly to an amino acid or polypeptide that stabilizes the active agent, primarily in the stomach, through conformational protection (see, e.g., Piccariello et al., U.S. Pat. No. 6,716,452, and U.S. Application Publication No. 2004/0127397 and 2004/0063628, each of which is incorporated by reference herein in its entirety). Piccariello et al. disclose conjugates in which a drug, such as propofol, can be covalently bonded directly to the N-terminus, the C-terminus or an amino acid side chain of a carrier peptide. In certain applications, the polypeptide can stabilize the drug in the gastrointestinal tract through conformational protection and/or can act as a substrate for transporters such as PEPT transporters.

These prodrugs, which can provide enhanced oral bioavailability of propofol, are distinguishable from propofol prodrugs having promoieties that provide enhanced aqueous solubility of propofol for intravenous administration. Propofol is widely used as a hypnotic sedative for intravenous administrating in the induction and maintenance of anesthesia or sedation in humans and animals. Propofol prodrugs with enhanced aqueous solubility for intravenous administration are disclosed, for example, by Stella et al., U.S. Patent Nos. 6,204,257 and 6,872,838, and U.S. Application Publication No. 2005-0090431; Marappan et al., 2005/0234050; and Wingard et al., 2005/0203068.

Examples of propofol prodrugs capable of providing an increased oral bioavailability of propofol relative to the oral bioavailability of propofol in which propofol is bonded to an amino acid or small peptide via a linkage are disclosed in Gallop et al., U.S. Application Publication No. 2005/0004381; Gallop et al., U.S. Application Publication No. 2005/0107385; Xu et al., U.S. Application Publication No. 2006/0041011; Xu et al., U.S. Application Publication No. 2006/0100160; and Xu et al., U.S. Application Publication No. 2005/0265609, each of which is incorporated by reference herein in its entirety.

In certain embodiments, a prodrug of propofol has the structure of Formula (I) as disclosed in Gallop et al., U.S. Application Publication No. 2005/0004381:

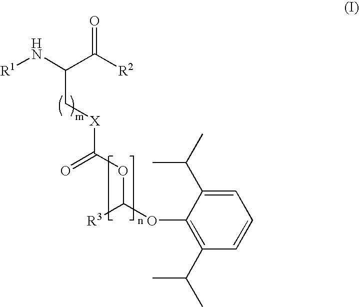

a pharmaceutically acceptable salt thereof, or a pharmaceutically acceptable solvate of any of the foregoing, wherein:

X is selected from bond, —CH₂—, —NR¹¹—, —O—, and —S—;

m is selected from 1 and 2;

n is selected from 0 and 1;

R¹ is selected from hydrogen, [R⁵NH(CHR⁴)ₚC(O)]—, R⁶—, R⁶C(O)—, and R⁶OC(O)—;

R² is selected from —OR⁷, and —[NR⁸(CHR⁹)_{q}C(O)OR⁷];

p and q are independently selected from 1 and 2;

R³ is selected from hydrogen, alkyl, substituted alkyl, alkoxycarbonyl, aryl, substituted aryl, arylalkyl, carbamoyl, substituted carbamoyl, cycloalkyl, substituted cycloalkyl, cycloheteroalkyl, heteroaryl, substituted heteroaryl, and heteroarylalkyl;

each R⁴ is independently selected from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl, or when R⁴ and R⁵ are bonded to adjacent atoms then R⁴ and R⁵ together with the atoms to which they are bonded form a cycloheteroalkyl or substituted cycloheteroalkyl ring;

R⁵ is selected from hydrogen, R⁶—, R⁶C(O)—, and R⁶OC(O)—;

R⁶ is selected from alkyl, substituted alkyl, aryl, substituted aryl, arylalkyl, substituted arylalkyl, cycloalkyl, substituted cycloalkyl, cycloheteroalkyl, heteroaryl, substituted heteroaryl, and heteroarylalkyl;

R⁷ is selected from hydrogen, alkyl, substituted alkyl, aryl, substituted aryl, arylalkyl, substituted arylalkyl, cycloalkyl, substituted cycloalkyl, cycloheteroalkyl, heteroaryl, substituted heteroaryl, and heteroarylalkyl;

R⁸ is selected from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl;

each R⁹ is independently selected from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl, or when R⁸ and R⁹ are bonded to adjacent atoms then R⁸ and R⁹ together with the atoms to which they are bonded form a cycloheteroalkyl or substituted cycloheteroalkyl ring; and

R¹¹ is selected from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl;

with the provisos that:

when R¹ is [R⁵NH(CHR⁴)ₚC(O)]— then R² is —OR⁷; and

when R² is —[NR⁸(CHR⁹)_{q}C(O)OR⁷] then R¹ is not [R⁵NH(CHR⁴)ₚC(O)]—.

In certain embodiments, a prodrug of propofol has the structure of Formula (II) as disclosed in Gallop et al., U.S. Application Publication No. 2005/0107385:

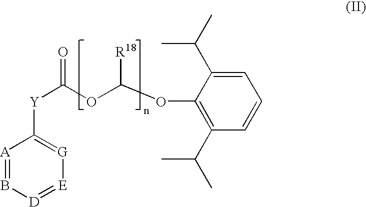

a pharmaceutically acceptable salt thereof, or a pharmaceutically acceptable solvate of any of the foregoing, wherein:

n is selected from 0 and 1;

Y is selected from a bond, CR¹R², NR³, O, and S;

A is selected from CR⁴ and N;

B is selected from CR⁵ and N;

D is selected from CR⁶ and N;

E is selected from CR⁷ and N;

G is selected from CR⁸ and N;

R¹⁸ is selected from hydrogen, alkyl, substituted alkyl, alkoxycarbonyl, aryl, substituted aryl, arylalkyl, carbamoyl, substituted carbamoyl, cycloalkyl, substituted cycloalkyl, cycloheteroalkyl, heteroaryl, substituted heteroaryl, and heteroarylalkyl;

R¹ and R² are independently selected from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl;

R³ is selected from hydrogen, alkyl, substituted alkyl, aryl, arylalkyl, cycloalkyl, and heteroaryl;

R⁴ is selected from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]ₖZ(CR⁹R¹⁰)ᵣCO₂R¹¹;

R⁵ is selected from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]ₖZ(CR⁹R¹⁰)ᵣCO₂R¹¹;

R⁶ is selected from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]ₖZ(CR⁹R¹⁰)ᵣCO₂R¹¹;

R⁷ is selected from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]ₖZ(CR⁹R¹⁰)ᵣCO₂R¹¹;

R⁸ is selected from hydrogen, alkyl, substituted alkyl, alkoxy, substituted alkoxy, alkoxycarbonyl, aryl, substituted aryl, arylalkyl, carboxyl, cycloalkyl, substituted cycloalkyl, cycloheteroalkyl, halogen, heteroaryl, substituted heteroaryl, heteroarylalkyl, hydroxyl, and —W[C(O)]ₖZ(CR⁹R¹⁰)ᵣCO₂R¹¹;

W is selected from a bond, —CR¹²R¹³, —NR¹⁴, O, and S;

Z is selected from —CR¹⁵R¹⁶, —NR¹⁷, O, and S;

k is selected from 0 and 1;

r is selected from 1, 2, and 3;

each of R⁹, R₁₀, R¹¹, R¹², R¹³, R¹⁵, and R¹⁶ is independently selected from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl; and

R¹⁴ and R¹⁷ are independently selected from hydrogen, alkyl, substituted alkyl, aryl, arylalkyl, cycloalkyl, and heteroaryl;

with the provisos that:
at least one of A, B, D, E, and G is not N;
one and only one of R⁴, R⁵, R⁶, k⁷, or R⁸ is —W[C(O)]ₖZ(CR⁹R¹⁰)ᵣCO₂R¹¹; and

if k is 0 then W is a bond.

In certain embodiments, a prodrug of propofol has the structure of Formula (III) as disclosed in Xu et al., U.S. Application Publication No. 2006/0041011:

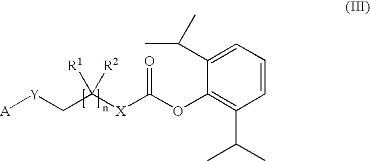

a pharmaceutically acceptable salt thereof, or a pharmaceutically acceptable solvate of any of the foregoing, wherein:

each R¹ and R² is independently selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R¹ and R² together with the carbon atom to which they are bonded form a cycloalkyl, substituted cycloalkyl, cycloheteroalkyl, or substituted cycloheteroalkyl ring;

A is selected from hydrogen, acyl, substituted acyl, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or A, Y, and one of R¹ and R² together with the atoms to which they are bonded form a cycloheteroalkyl or substituted cycloheteroalkyl ring;

Y is selected from —O— and —NR³—;

R³ is selected from hydrogen, alkyl, substituted alkyl, arylalkyl, and substituted arylalkyl;

n is an integer from 1 to 5;

X is selected from —NR⁴—, —O—, —CH₂, and —S—; and

R⁴ is selected from hydrogen, alkyl, substituted alkyl, arylalkyl, and substituted arylalkyl.

In certain embodiments, a prodrug of propofol has the structure of Formula (IV) as disclosed in Xu et al., U.S. Application Publication No. 2006/0041011:

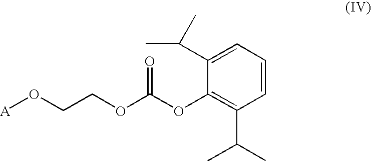

or a pharmaceutically acceptable salt thereof, or a pharmaceutically acceptable solvate of any of the foregoing, wherein:

A is selected from hydrogen and [H₂NCHR⁵C(O)]—; and

R⁵ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R⁵ and the alpha amino group together with the atoms to which they are bonded form a cycloheteroalkyl or substituted cycloheteroalkyl ring.

In certain embodiments, a prodrug of propofol has the structure of Formula (V) as disclosed in Xu et al., U.S. Application Publication 1 No. 2006/0041011:

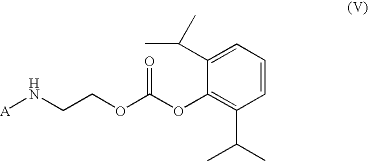

a pharmaceutically acceptable salt thereof, or a pharmaceutically acceptable solvate of any of the foregoing, wherein:

A is selected from hydrogen, [H₂NCHR⁵C(O)]— and —C(O)OR⁶;

R⁵ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R⁵ and the alpha amino group together with the atoms to which they are bonded form a cycloheteroalkyl or substituted cycloheteroalkyl ring; and

R⁶ is selected from alkyl, substituted alkyl, aryl, substituted aryl, arylalkyl, and substituted arylalkyl.

In certain embodiments, a prodrug of propofol has the structure of Formula (VI) as disclosed in Xu et al., U.S. Application Publication No. 2006/0041011:

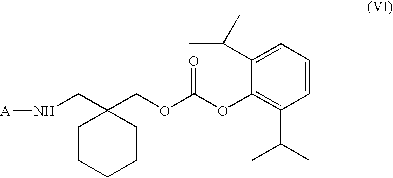

a pharmaceutically acceptable salt thereof, or a pharmaceutically acceptable solvate of any of the foregoing, wherein:

A is selected from hydrogen and [H₂NCHR⁵C(O)]—; and

R⁵ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R⁵ and the alpha amino group together with the atoms to which they are bonded form a cycloheteroalkyl or substituted cycloheteroalkyl ring.

In certain embodiments, a prodrug of propofol has the structure of Formula (VII) as disclosed in Xu et al., U.S. Application Publication No. 2006/0041011:

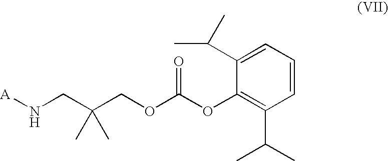

a pharmaceutically acceptable salt thereof, or a pharmaceutically acceptable solvate of any of the foregoing, wherein:

A is selected from hydrogen and [H₂NCHR⁵C(O)]—; and

R⁵ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R⁵ and the alpha amino group together with the atoms to which they are bonded form a cycloheteroalkyl or substituted cycloheteroalkyl ring.

In certain embodiments, a prodrug of propofol has the structure of Formula (VIII) as disclosed in Xu et al., U.S. Application Publication No. 2006/0041011:

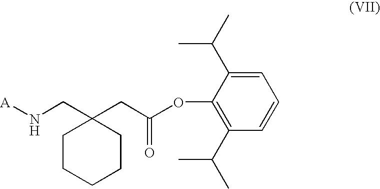

a pharmaceutically acceptable salt thereof, or a pharmaceutically acceptable solvate of any of the foregoing, wherein:

A is selected from hydrogen and [H₂NCHR⁵C(O)]—; and

R⁵ is selected from hydrogen, C₁₋₆ alkyl, substituted C₁₋₆ alkyl, C₅₋₇ aryl, substituted C₅₋₇ aryl, C₆₋₁₁ arylalkyl, substituted C₆₋₁₁ arylalkyl, C₁₋₆ heteroalkyl, substituted C₁₋₆ heteroalkyl, C₅₋₇ heteroaryl, substituted C₅₋₇ heteroaryl, C₆₋₁₁ heteroarylalkyl, and substituted C₆₋₁₁ heteroarylalkyl, or R⁵ and the alpha amino group together with the atoms to which they are bonded form a C₅₋₇ cycloheteroalkyl or substituted C₅₋₇ cycloheteroalkyl ring.

In certain embodiments, a prodrug of propofol has the structure of Formula (IX) as disclosed in Xu et al., U.S. Application Publication No. 2006/0041011:

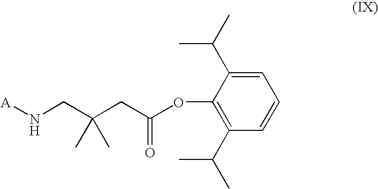

a pharmaceutically acceptable salt thereof, or a pharmaceutically acceptable solvate of any of the foregoing, wherein:

A is selected from hydrogen and [H₂NCHR⁵C(O)]—; and

R⁵ is selected from hydrogen, C₁₋₆ alkyl, substituted C₁₋₆ alkyl, C₅₋₇ aryl, substituted C₅₋₇ aryl, C₆₋₁₁ arylalkyl, substituted C₆₋₁₁ arylalkyl, C₁₋₆ heteroalkyl, substituted C₁₋₆ heteroalkyl, C₅₋₇ heteroaryl, substituted C₅₋₇ heteroaryl, C₆₋₁₁ heteroarylalkyl, and substituted C₆₋₁₁ heteroarylalkyl, or R⁵ and the alpha amino group together with the atoms to which they are bonded form a C₅₋₇ cycloheteroalkyl or substituted C₅₋₇ cycloheteroalkyl ring.

In certain embodiments, a prodrug of propofol has the structure of Formula (X) as disclosed in Xu et al., U.S. Application Publication No. 2006/0041011:

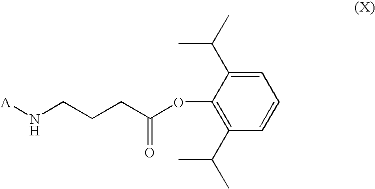

a pharmaceutically acceptable salt thereof, or a pharmaceutically acceptable solvate of any of the foregoing, wherein:

A is selected from hydrogen and [H₂NCHR⁵C(O)]—; and

R⁵ is selected from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R⁵ and the alpha amino group together with the atoms to which they are bonded form a cycloheteroalkyl or substituted cycloheteroalkyl ring.

In certain embodiments, a prodrug of propofol has the structure of Formula (XI) as disclosed in Xu et al., U.S. Application Publication No. 2006/0100160:

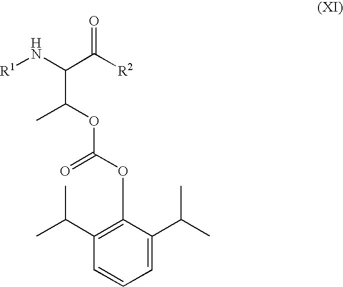

a pharmaceutically acceptable salt thereof, or a pharmaceutically acceptable solvate of any of the foregoing, wherein:

R¹ is selected from hydrogen, [R⁵NH(CHR⁴)ₚC(O)]—, R⁶—, R⁶C(O)—, and R⁶OC(O)—;

R² is selected from —OR⁷ and —[NR⁸(CHR⁹)_{q}C(O)OR⁷];

p and q are independently selected from 1 and 2;

each R⁴ is independently selected from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl, or when R⁴ and R⁵ are bonded to adjacent atoms then R⁴ and R⁵ together with the atoms to which they are bonded form a cycloheteroalkyl or substituted cycloheteroalkyl ring;

R⁵ is selected from hydrogen, R⁶—, R⁶C(O)—, and R⁶OC(O)—;

R⁶ is selected from alkyl, substituted alkyl, aryl, substituted aryl, arylalkyl, substituted arylalkyl, cycloalkyl, substituted cycloalkyl, cycloheteroalkyl, heteroaryl, substituted heteroaryl, and heteroarylalkyl;

R⁷ is selected from hydrogen, alkyl, substituted alkyl, aryl, substituted aryl, arylalkyl, substituted arylalkyl, cycloalkyl, substituted cycloalkyl, cycloheteroalkyl, heteroaryl, substituted heteroaryl, and heteroarylalkyl;

R⁸ is selected from hydrogen, alkyl, substituted alkyl, aryl, substituted aryl, arylalkyl, cycloalkyl, substituted cycloalkyl, cycloheteroalkyl, heteroaryl, substituted heteroaryl, and heteroarylalkyl; and

each R⁹ is independently selected from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl, or when R⁸ and R⁹ are bonded to adjacent atoms then R⁸ and R⁹ together with the atoms to which they are bonded form a cycloheteroalkyl or substituted cycloheteroalkyl ring;

with the proviso that when R² is —[NR⁸(CUR⁹)_{q}C(O)OR⁷] then R¹ is not [R⁵NH(CHR⁴)ₚC(O)]—.

In certain embodiments, a prodrug of propofol has the structure of Formula (XII):

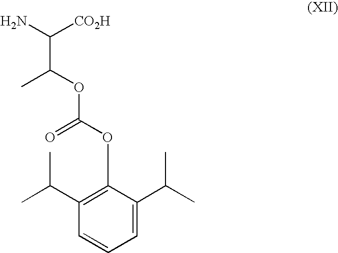

a pharmaceutically acceptable salt thereof, or a pharmaceutically acceptable solvate of any of the foregoing.

In certain embodiments of a compound of Formula (XII), the α-carbon of the amino acid residue is of the L-configuration. In certain embodiments of a compound of Formula (XII), the α-carbon of the amino acid residue is of the D-configuration.

In certain embodiments, a prodrug of propofol has the structure of Formula (XIII) as disclosed in Xu et al., U.S. Application Publication No. 2005/0265609:

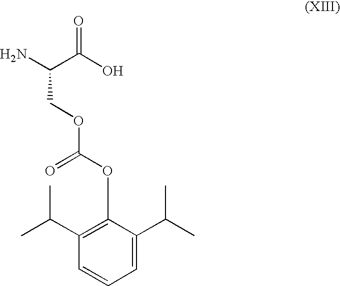

a pharmaceutically acceptable salt thereof, or a pharmaceutically acceptable solvate of any of the foregoing.

In certain embodiments, a prodrug of propofol of Formula (XIII) can be a crystalline form of 2-amino-3-(2,6-diisopropyl-phenoxycarbonyloxy)-propanoic acid or pharmaceutically acceptable salts or solvates thereof. In certain embodiments, a prodrug of propofol of Formula (XIII) can be a crystalline form of (S)-2-amino-3-(2,6-diisopropylphenoxycarbonyloxy)-propanoic acid or pharmaceutically acceptable salts thereof, or pharmaceutically acceptable solvates thereof. In certain embodiments, a prodrug of propofol can be crystalline 2-amino-3-(2,6-diisopropylphenoxycarbonyloxy)-propanoic acid hydrochloride. In certain embodiments, a prodrug of propofol can be crystalline (S)-2-amino-3-(2,6-diisopropylphenoxycarbonyloxy)-propanoic acid hydrochloride. In certain embodiments, a prodrug of propofol can be crystalline (S)-2-amino-3-(2,6-diisopropylphenoxy-carbonyloxy)-propanoic acid hydrochloride having characteristic peaks (2θ) at 5.1°±0.2°, 9.7°±0.2°, 11.0°±0.2°, 14.1°±0.2°, 15.1°±0.2°, 15.8°±0.2°, 17.9°±0.2°, 18.5°±0.2°, 19.4°±0.2°, 20.1°±0.2°, 21.3°±0.2°, 21.7°±0.2°, 22.5°±0.2°, 23.5°±0.2°, 24.4°±0.2°, 25.1±0.2°, 26.8°±0.2°, 27.3°±0.2°, 27.8°±0.2°, 29.2°±0.2°, 29.6°±0.2°, 30.4°±0.2°, and 33.4°±0.2° in diffraction pattern. In certain embodiments, a prodrug of propofol can be crystalline (S)-2-amino-3-(2,6-diisopropylphenoxycarbonyloxy)-propanoic acid hydrochloride having characteristic peaks (2θ) at 5.1°±0.2°, 9.7°±0.2°, 11.0°±0.2°, 14.1°±0.2°, 15.1°±0.2°, 15.8°±0.2°, 17.9°±0.2°, 18.5°±0.2°, 20.1°±0.2°, 22.5°±0.2°, 23.5°±0.2°, 25.1°±0.2°, 29.2°+0.2°, 29.6°±0.2°, and 33.4°±0.2° in an X-ray powder diffraction pattern.

In certain embodiments, a prodrug of propofol can be crystalline 2-amino-3-(2,6-diisopropylphenoxycarbonyloxy)-propanoic acid hydrochloride having a melting point from about 180° C. to about 200° C. In certain embodiments, a prodrug of propofol can be crystalline 2-amino-3-(2,6-diisopropylphenoxycarbonyloxy)-propanoic acid hydrochloride having a melting point from about 185° C. to about 195° C. In certain embodiments, a prodrug of propofol can be crystalline (S)-2-amino-3-(2,6-diisopropylphenoxycarbonyloxy)-propanoic acid hydrochloride having a melting point from about 188° C. to about 189° C.

In certain embodiments, a prodrug of propofol can be crystalline 2-amino-3-(2,6-diisopropylphenoxycarbonyloxy)-propanoic acid mesylate. In certain embodiments, a prodrug of propofol can be crystalline (S)-2-amino-3-(2,6-diisopropylphenoxycarbonyloxy)-propanoic acid mesylate. In certain embodiments, a prodrug of propofol can be crystalline (S)-2-amino-3-(2,6-diisopropylphenoxycarbonyloxy)-propanoic acid mesylate having characteristic peaks (2θ) at 4.2°±0.1°, 11.7°±0.1°, 12.1°±0.1°, 12.6°±0.1°, 16.8°±0.1°, 18.4°±0.2°, 21.0°±0.1°, 22.3°±0.1°, 22.8°±0.2°, 24.9°±0.2°, 25.3°±0.1°, 26.7°±0.2°, and 29.6°±0.1° in an X-ray powder diffraction pattern. In certain embodiments, a prodrug of propofol can be crystalline (S)-2-amino-3-(2,6-diisopropylphenoxycarbonyloxy)-propanoic acid mesylate having characteristic peaks (2θ) at 4.2°±0.1°, 12.6°±0.1°, 16.8°±0.1°, 21.0°±0.1°, 25.3°±0.1°, 2 and 29.6°±0.1° in an X-ray powder diffraction pattern.

In certain embodiments, a prodrug of propofol can be crystalline 2-amino-3-(2,6-diisopropylphenoxycarbonyloxy)-propanoic acid mesylate having a melting point from about 156° C. to about 176° C. In certain embodiments, a prodrug of propofol can be crystalline 2-amino-3-(2,6-diisopropylphenoxycarbonyloxy)-propanoic acid mesylate having a melting point from about 161° C. to about 172° C. In certain embodiments, a prodrug of propofol can be crystalline (S)-2-amino-3-(2,6-diisopropylphenoxycarbonyloxy)-propanoic acid mesylate having a melting point from about 166° C. to about 167° C.

Propofol prodrugs of Formulae (I)-(XIII) can be administered orally and transported across cells (i.e., enterocytes) lining the lumen of the gastrointestinal tract. Certain of the compounds of structural Formulae (I)-(XIII) may be substrates for the proton-coupled intestinal peptide transport system (PEPT1) (Leibach et al., Annu. Rev. Nutr. 1996, 16, 99-119), which mediates the cellular uptake of small intact peptides consisting of two or three amino acids that are derived from the digestion of dietary proteins. In the intestine, where small peptides are not effectively absorbed by passive diffusion, PEPT1 may act as a vehicle for the effective uptake of small peptides across the apical membrane of the gastric mucosa including propofol prodrugs of Formulae (I)-(XIII).

Methods for determining whether propofol prodrugs of Formulae (I)-(XIII) serve as substrates for the PEPT1 transporter are disclosed, for example, in see Xu et al., U.S. Application Publication No. 2006/0100160. In vitro systems using cells engineered to heterologously express the PEPT1 transport system or cell-lines that endogenously express the transporter (e.g. Caco-2 cells) may be used to assay transport of compounds of Formulae (I)-(XIII) by the PEPT1 transporter. Standard methods for evaluating the enzymatic conversion of a propofol prodrug to propofol in vitro are disclosed, for example, in Xu et al., U.S. Application Publication No. 2006/0100160.

Propofol prodrugs of Formula (I)-(XIII) can exhibit sufficient stability to enzymatic and/or chemical degradation in the gastrointestinal tract resulting in enhanced bioavailability. Propofol prodrugs of Formula (I)-(XIII) can also exhibit enhanced passive and/or active gastrointestinal absorption compared to propofol

Oral administration of propofol prodrugs to animals is described in Xu et al., U.S. Application Publication Nos. 2006/0041011, 2006/0100160, and 2005/0265609, and illustrates that actively transported propofol prodrugs can afford significant enhancement in the oral bioavailability of propofol relative to the oral bioavailability of propofol when administered in an equivalent dosage form. In certain embodiments, a prodrug of propofol provides greater than 10% absolute oral bioavailability of propofol, i.e., compared to the bioavailability of propofol following intravenous administration of an equimolar dose of propofol itself. A prodrug of propofol that provides at least about 10 times higher oral bioavailability of propofol compared to the oral bioavailability of propofol itself, and in certain embodiments, at least about 40 times higher oral bioavailability of propofol compared to the oral bioavailability of propofol itself when orally administered in an equivalent dosage form (see, e.g., Xu et al., U.S. Application Publication 1 No. 2006/0100160; and Xu et al., U.S. Application Publication No. 2005/0265609).

Methods of synthesizing prodrugs of propofol of Formula (I) are disclosed in Gallop et al., U.S. Application Publication No. 2005/0004381. Methods of synthesizing prodrugs of propofol of Formula (II) are disclosed in Gallop et al., U.S. Application Publication No. 2005/0107385. Methods of synthesizing prodrugs of propofol of Formulae (III)-(X) are disclosed in Xu et al., U.S. Application Publication No. 2006/0041011. Methods of synthesizing prodrugs of propofol of Formulae (XI)-(XII) are disclosed in Xu et al., U.S. Application Publication No. 2006/0100160. Methods of synthesizing and crystallizing prodrugs of propofol of Formula (XIII) are disclosed in Xu et al., U.S. Application Publication No. 2005/0265609.

In certain embodiments, pharmaceutical compositions provided by the present disclosure comprise a form of propofol selected from a propofol prodrug, and a propofol tight-ion pair complex. In certain embodiments, a pharmaceutical composition comprises a propofol prodrug, and in certain embodiments the propofol prodrug is selected from a compound of Formula (I) to Formula (XIII) a pharmaceutically acceptable salt of any of the foregoing, a pharmaceutically acceptable solvate of any of the foregoing, or a combination of any of the foregoing. In certain embodiments, a propofol prodrug is a compound of Formula (XIII), and in certain embodiments a propofol prodrug is (S)-2-amino-3-(2,6-diisopropylphenoxycarbonyloxy)-propanoic acid, a pharmaceutically acceptable salt thereof, or a pharmaceutically acceptable solvate of any of the foregoing. In certain embodiments, a pharmaceutical composition comprises a 5-HT₃ receptor antagonist, a corticosteroid, and (S)-2-amino-3-(2,6-diisopropylphenoxycarbonyloxy)-propanoic acid, a pharmaceutically acceptable salt thereof, or a pharmaceutically acceptable solvate of any of the foregoing.

In certain embodiments, a pharmaceutical composition comprises a serotonin 5-HT₃ receptor antagonist selected from alosetron, azasetron, bemesetron, cilansetron, dolasetron, granisetron, indeseetron, itasetron, ondansetron, palonosetron, ramosetron, tropisetron, and zatoseteron; a corticosteroid selected from dexamethasone and methylpredisolone; and (S)-2-amino-3-(2,6-diisopropylphenoxycarbonyloxy)-propanoic acid, or a pharmaceutically acceptable salt thereof, or a pharmaceutically acceptable solvate of any of the foregoing. In certain embodiments, a pharmaceutical composition comprises ondansetron, dexamethasone, and (S)-2-amino-3-(2,6-diisopropylphenoxycarbonyloxy)-propanoic acid.

In certain embodiments, a pharmaceutical composition can include an adjuvant that facilitates absorption of an anti-emetic compound and/or highly orally bioavailable form of propofol through the gastrointestinal epithelia. Such enhancers can, for example, open the tight-junctions in the gastrointestinal tract or modify the effect of cellular components, such as p-glycoprotein and the like. Suitable enhancers include alkali metal salts of salicylic acid, such as sodium salicylate, caprylic or capric acid, such as sodium caprylate or sodium caprate, and the like. Enhancers include, for example, the bile salts, such as sodium deoxycholate. Various p-glycoprotein modulators are described in U.S. Pat. Nos. 5,112,817 and 5,643,909. Various absorption enhancing compounds and materials are described in U.S. Pat. No.5,824,638, and U.S. Application Publication No.2006/0046962. Other adjuvants that enhance permeability of cellular membranes include resorcinol, surfactants, polyethylene glycol, and bile acids. In certain embodiments, a permeation enhance may enhance the passive permeation of the form of the anti-emetic compound and/or highly orally bioavailable form of propofol from the gastrointestinal lumen into the blood.

In certain embodiments, a pharmaceutical composition can include an adjuvant that reduces enzymatic degradation of the anti-emetic compound and/or highly orally bioavailable form of propofol in the gastrointestinal tract and/or the systemic circulation. Microencapsulation using encapsulants such as protenoid microspheres, liposomes, or polysaccharides has also been shown effective in abating enzymatic degradation of orally ingested compounds.

Pharmaceutical composition can also include one or more pharmaceutically acceptable vehicles, including excipients, adjuvants, carriers, diluents, binders, lubricants, disintegrants, colorants, stabilizers, surfactants, fillers, buffers, and the like. Vehicles can be selected to alter the porosity and permeability of a pharmaceutical composition, alter hydration and disintegration properties, control hydration, enhance manufacturability, etc.

Pharmaceutical compositions can be produced using standard procedures (see, e.g., “Remington's The Science and Practice of Pharmacy,” 21^{st} Edition, Lippincott, Williams & Wilcox, 2005).

Pharmaceutical compositions provided by the present disclosure comprise an anti-emetic compound and a highly orally bioavailable form of propofol. In certain embodiments, the pharmaceutical compositions are formulated for oral administration. Pharmaceutical compositions formulated for oral administration can provide for uptake of the anti-emetic compound and highly orally bioavailable form of propofol throughout the gastrointestinal tract or in a particular region or regions of the gastrointestinal tract. In certain embodiments, a pharmaceutical composition is formulated to enhance uptake of the highly orally bioavailable form of propofol from the upper gastrointestinal tract, and in certain embodiments, from the small intestine. Such compositions can be prepared in a manner known in the pharmaceutical art and may further comprise, in addition to a first anti-emetic compound and a highly orally bioavailable form of propofol, one or more pharmaceutically acceptable vehicles, permeability enhancers, and/or a second anti-emetic compound.

A pharmaceutical composition can comprise a therapeutically effective amount of an anti-emetic compound and a therapeutically effective amount of a highly orally bioavailable form of propofol. In certain embodiments, a pharmaceutical composition can comprise less than a therapeutically effective amount of an anti-emetic compound, less than a therapeutically effective amount of a highly orally bioavailable form of propofol, or less than a therapeutically effective amount of both an anti-emetic compound and a highly orally bioavailable form of propofol. In embodiments in which the pharmaceutical composition comprises less than a therapeutically effective amount of an anti-emetic compound, a highly orally bioavailable form of propofol, or both, the combined amounts of the anti-emetic compound and the highly orally bioavailable form of propofol provide a therapeutically effective amount.

In certain embodiments, a pharmaceutical composition can include more than one anti-emetic compound and/or more than one highly orally bioavailable form of propofol.

Pharmaceutical compositions may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of compounds disclosed herein into preparations, which can be used pharmaceutically.

Pharmaceutical compositions provided by the present disclosure can provide therapeutic or prophylactic levels of an anti-emetic compound and propofol upon oral administration to a patient. The promoiety of a highly orally bioavailable form of propofol may be cleaved in vivo either chemically and/or enzymatically to release the drug, propofol. One or more enzymes present in the blood, liver, brain, or any other suitable tissue of a mammal may enzymatically cleave the promoiety of the administered propofol prodrugs. In certain embodiments, propofol remains conjugated to the promoiety during transit across the intestinal mucosal barrier to provide protection from presystemic metabolism. In certain embodiments, a highly orally bioavailable form of propofol is essentially not metabolized to propofol within enterocytes, but is metabolized to the parent drug, propofol, within the systemic circulation. Cleavage of the promoiety of the propofol prodrug after absorption by the gastrointestinal tract may allow the prodrugs to be absorbed into the systemic circulation either by active transport, passive diffusion, or by a combination of both active and passive processes.

In certain embodiments, pharmaceutical compositions comprise an amount of a highly orally bioavailable form of propofol capable of providing a plasma concentration of propofol in a patient from about 10 ng/mL to less than a concentration that induces sedation in the patient for a continuous time period of at least about 4 hours following oral administration, for a time period of at least about 8 hours, for a time period of at least about 12 hours, for a time period of at least about 16 hours, and in certain embodiments for a time period of at least about 20 hours. In certain embodiments, pharmaceutical compositions comprise an amount of a highly orally bioavailable form of propofol capable of providing a plasma concentration of propofol in a patient from about 100 ng/mL to less than a concentration that induces sedation in the patient for a continuous time period of at least about 4 hours following oral administration of the dosage form, for a time period of at least about 8 hours, for a time period of at least about 12 hours, for a time period of at least about 16 hours, and in certain embodiments for a time period of at least about 20 hours. By continuous time period is meant that the concentration of propofol is maintained above a prescribed minimum concentration, or within a prescribed concentration range throughout the indicated time period.

Pharmaceutical compositions provided by the present disclosure may further comprise, in addition to a first anti-emetic compound and a highly orally bioavailable form of propofol, one or more additional anti-emetic compounds. In certain embodiments, a first anti-emetic compound is a serotonin 5-HT₃ receptor antagonist such as dolasetron, granisetron, ondansetron, palonosetron, itasetron, tropisetron, and ramosetron. In certain embodiments, the first anti-emetic compound is ondansetron.

In certain embodiments, a highly orally bioavailable form of propofol is selected from a propofol tight-ion pair and a propofol prodrug. In certain embodiments, a highly orally bioavailable form of propofol is a propofol prodrug. In certain embodiments, a propofol prodrug is selected from a compound of Formula (I) to Formula (XII) or a pharmaceutically acceptable salt thereof, or a pharmaceutically acceptable solvate of any of the foregoing. In certain embodiments, the prodrug of propofol is a compound of Formula (XIII) or a pharmaceutically acceptable salt thereof, or a pharmaceutically acceptable solvate of any of the foregoing. In certain embodiments, an additional anti-emetic compound is a corticosteroid such as dexamethasone or metylprednisolone. In certain embodiments, the pharmaceutical composition comprises a serotonin 5-HT₃ receptor antagonist selected from dolasetron, granisetron, ondansetron, palonosetron, itasetron, tropisetron, and ramosetron, a propofol prodrug of Formula (XIII), and optionally a corticosteroid selected from dexamethasone and methylprednisolone.

In certain embodiments, pharmaceutical composition may further comprise substances to enhance, modulate and/or control release, bioavailability, therapeutic efficacy, therapeutic potency, stability, and the like. For example, to enhance therapeutic efficacy a drug may be co-administered with one or more active agents to increase the absorption or diffusion of the drug from the gastrointestinal tract, or to inhibit degradation of the drug in the systemic circulation. In certain embodiments, a drug may be co-administered with active agents having pharmacological effects that enhance the therapeutic efficacy of the drug.

Present pharmaceutical compositions can take the form of solutions, suspensions, emulsions, tablets, lozenges, pills, pellets, granules, capsules, capsules containing liquids, capsules containing solids, capsules containing particulates, powders, emulsions, suspensions, or any other form suitable for oral administration. Examples of suitable pharmaceutical vehicles have been described in the art (see “Remington's Pharmaceutical Sciences,” Lippincott Williams & Wilkins, 21st edition, 2005). Orally administered compositions may contain one or more optional agents, for example, sweetening agents such as fructose, aspartame or saccharin, flavoring agents such as peppermint, oil of wintergreen, or cherry coloring agents, and preserving agents, to provide a pharmaceutically palatable preparation. Moreover, when in tablet or pill form, a composition may be coated to delay disintegration and absorption in the gastrointestinal tract, thereby providing a sustained action over an extended period of time. Oral compositions may include standard vehicles such as mannitol, lactose, starch, magnesium stearate, sodium saccharine, cellulose, magnesium carbonate, etc. Such vehicles are preferably of pharmaceutical grade. For preparing solid compositions such as tablets, an anti-emetic compound and a highly orally bioavailable form of propofol can be mixed with a pharmaceutically acceptable vehicle to form a solid pre-formulation composition containing a homogeneous mixture in which the anti-emetic compound and a highly orally bioavailable form of propofol are dispersed evenly throughout the composition so that the composition can be readily subdivided into equally effective unit dosage forms such as tablets, pills, or capsules. The solid pre-formulation can then be subdivide into unit dosage forms. For oral liquid preparations such as, for example, suspensions, elixirs and solutions, suitable carriers, excipients, or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcarnitines, and the like may be added.

Pharmaceutical compositions provided by the present disclosure may be formulated so as to provide immediate, sustained, or delayed release of an anti-emetic compound and a prodrug of propofol after administration to the patient by employing procedures known in the art (see, e.g., Allen et al., “Ansel's Pharmaceutical Dosage Forms and Drug Delivery Systems,” 8th ed., Lippincott, Williams & Wilkins, August 2004).

In embodiments in which an anti-emetic compound and a highly orally bioavailable form of propofol are administered separately, the pharmaceutical compositions separately comprising each active agent can be provided in a kit form. A kit may include two separate pharmaceutical compositions—the first pharmaceutical composition comprising an anti-emetic compound and the second pharmaceutical composition comprising a highly orally bioavailable form of propofol. A kit may include a container for containing the separate compositions such as a divided bottle or a divided foil packet. A kit may also include directions for administering the separate compositions. A kit can be particularly advantageous when the separate compositions are administered in different dosage forms, e.g., oral and parenteral, are administered at different dosage intervals, or when titration of the anti-emetic compound and the highly orally bioavailable form of propofol is desired by the prescribing physician.

### Dosage Forms

Pharmaceutical compositions provided by the present disclosure may be formulated in a unit dosage form. Unit dosage form refers to a physically discrete unit suitable as unitary dosage for patients undergoing treatment, with each unit containing a predetermined quantity of an anti-emetic compound and a highly orally bioavailable form of propofol calculated to produce the intended therapeutic effect.

A unit dosage form may be for a single daily dose or one of multiple daily doses, e.g., 2 to 4 times per day. When multiple daily doses are used, the unit dosage can be the same or different for each dose. One or more dosage forms may comprise a dose, which is administered to a patient at a single point in time or during a time interval.

In certain embodiments, a therapeutically effective dose of a first anti-emetic compound comprises from about 0.01 mg to about 1,000 mg per day, from about 0.1 mg to about 500 mg per day, from about 1 mg to about 50 mg per day, and in certain embodiments, from about 2 to about 25 mg per day. A dose may be administered in a single dosage form or in multiple dosage forms, for example from 1 to 4 or more times per day. When multiple dosages are used, the amount of a first anti-emetic compound in each dosage may be the same or different.

In certain embodiments wherein a pharmaceutical composition provided by the present disclosure further comprises a second anti-emetic compound such as a corticosteroid, a therapeutically effective dose of the second anti-emetic compound may comprise from about 0.01 mg to about 100 mg per day, from about 0.1 mg to about 500 mg per day, from about 1 mg to about 50 mg per day, and in certain embodiments, from about 2 to about 25 mg per day. Examples of anti-emetic compounds and examples of therapeutically effective daily (d) dosages for treating emesis include: alosetron (0.5-4 mg/day), alprazolam (0.75-15 mg/day), aprepitant (50-200 mg/day), dexamethasone (2-30 mg/day), dimenhydrinate (10-400 mg/day), diphenhydramine (10-100 mg/day), dolasetron (8-300 mg/day), dronabinol (10-120 mg/day), droperidol (0.25-3 mg/day), granisteron (0.25-4 mg/day), haloperidol (2-20 mg/day), lorazepam (0.25-4 mg/day), metoclopramide (50-300 mg/day), olanzapine (1-20 mg/day), ondansetron (2-30 mg/day), palonosetron (0.1-2 mg/day), proclorperazine (2-100 mg/day), promethazine (5-50 mg/day), and tropisetron (1-10 mg/day). A therapeutically effective dose of anti-emetic compound will depend, at least in part, on the type of emesis being treated and the stage of the treatment regimen. For example, a higher dose of an anti-emetic compound may be administered initially with lesser amounts administered at subsequent days during the treatment regimen. A therapeutically effective dose can also depend on the route of administration.

In certain embodiments, a therapeutically effective dose of an anti-emetic compound can provide a therapeutically effective plasma concentration of the anti-emetic compound following administration to a patient. A therapeutically effective plasma concentration can depend on the potency, pharmacokinetics, and the route of administering the anti-emetic compound, and on the type and severity of the emesis being treated. For example, an oral dose of 8 mg ondansetron provides an oral bioavailability of about 48-75%, a maximum plasma concentration of about 20-52 ng/mL, an AUC of about 101-351 ng·mL·h⁻¹, a time to Cₘₐₓ of 1-2.2 h, a terminal elimination half-life of 2.5-6.2 h (see Blum et al., Clinical Therapeutics 2003, 1407-1419; de Wit et al., Br J Cancer 1996, 74, 323-326; Colthup et al., J Pharmaceutical Sciences, 1991, 80(9), 868-871; Britto et al., Clin Pharmacol Ther 1997, 61, 228; Villikka et al., Clin Pharmacol Ther 1999, 65, 377-381; De Witte et al., 2001, 92, 1319-1321; Arcioni et al., Anesth Anal 2002, 94, 1553-57; Tramer et al., Anesthesiology 1997, 87, 1277-89; and Tramer et al., Br Medical J 1997, 314, 1088-92). An oral dose of 5 mg tropisetron provides an oral bioavailability of about 60-99%, a maximum plasma concentration of about 22 ng/mL, an AUC of about 230 ng·mL·h⁻¹, a time to Cₘₐₓ of 2 h, a terminal elimination half-life of 8.6 h (Yarker et al., Drugs 1994, 48, 761-93). An oral dose of 5-20 mg granisetron provides a maximum plasma concentration of about 14-68 ng/mL, an AUC of about 83-350 ng·mL·h⁻¹, a time to Cₘₐₓ of 1-2.7 h, a terminal elimination half-life of 1.5-5.7 h (Fuji et al., Anesthesia & Analgesia 1997, 85, 913-7). An oral dose of 25-200 mg dolasetron provides an oral bioavailability of about 70-89%, a maximum plasma concentration of about 267 ng/mL, an AUC of about 1,233 ng·mL·h⁻¹, a time to Cₘₐₓ of 1.4 h, a terminal elimination half-life of 5-10 h (Tramer et al., Anesthesiology 1997, 87, 1277-89). Thus, in certain embodiments, a therapeutically effective plasma concentration of ondansetron can be from about 5 ng/mL to about 50 ng/mL, a therapeutically effective plasma concentration of tropisetron can be from about 2 ng/mL to about 25 ng/mL, a therapeutically effective plasma concentration of granisetron can be from about 5 ng/mL to about 70 ng/mL, and a therapeutically effective plasma concentration of dolesetron can be from about 20 ng/mL to about 300 ng/mL,

In certain embodiments, a therapeutically effective dose of a highly orally bioavailable form of propofol may comprise from about 10 mg to about 5,000 mg-equivalents of propofol, from about 50 mg to about 2,000 mg-equivalents of propofol, and in certain embodiments from about 100 mg to about 1,000 equivalents of propofol.

In certain embodiments, a therapeutically effective dose of a highly orally bioavailable form of propofol may provide a plasma concentration of propofol from about 10 ng/mL to less than a sedative concentration, from about 10 ng/mL to about 1,000 ng/mL, and in certain embodiments, from about 200 ng/mL to about 1,000 ng/mL for a continuous period of time. In certain embodiments, a therapeutically effective dose of a highly orally bioavailable form of propofol may provide a plasma concentration of propofol that is therapeutically effective and that is less than a concentration effective in causing sedation in the patient, for example, less than about 1,500 ng/mL or less than about 2,000 ng/mL. In certain embodiments, a therapeutically effective dose of a highly orally bioavailable form of propofol may provide a plasma concentration of propofol that is therapeutically effective and that is less than a concentration effective for the maintenance of general anesthesia (e.g., a sub-hypnotic concentration), for example, less than about 3,000 ng/mL or less than about 10,000 ng/mL.

Controlled drug delivery systems can be designed to deliver a drug in such a way that the drug level is maintained within the therapeutic windows and effective and safe blood levels of each of the administered drugs are maintained for a period as long as the system continues to deliver the drug at a particular rate. Controlled drug delivery can produce substantially constant blood levels of a drug as compared to fluctuations observed with immediate release dosage forms. For some drugs, maintaining a constant bloodstream and tissue concentration throughout the course of therapy is the most desirable mode of treatment. Immediate release of these drugs can cause blood levels to peak above the level required to elicit the desired response, which wastes the drug and may cause or exacerbate toxic side effects. Controlled drug delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

In certain embodiments, an oral dosage form provided by the present disclosure can be a controlled release dosage form. Controlled delivery technologies can improve the absorption of a drug in a particular region or regions of the gastrointestinal tract. For example, a dosage form can be adapted to facilitate delivery of an anti-emetic compound and/or highly orally bioavailable form of propofol to the small intestine and/or to facilitate absorption of an anti-emetic compound and/or highly orally bioavailable form of propofol from the small intestine into the blood. In certain embodiments, a controlled delivery oral dosage form can facilitate absorption of an anti-emetic compound and/or highly orally bioavailable form of propofol primarily from the small intestine, primarily from the large intestine, or from both the small and large intestine.

The appropriate oral dosage form for a particular pharmaceutical composition provided by the present disclosure can depend, at least in part, on the gastrointestinal absorption properties of the anti-emetic compound and the highly orally bioavailable form of propofol, the stability of the anti-emetic compound and the highly orally bioavailable form of propofol in the gastrointestinal tract, the pharmacokinetics of the anti-emetic compound and the highly orally bioavailable form of propofol, and the intended therapeutic profile. An appropriate controlled release oral dosage form can be selected for a particular combination of anti-emetic compound and highly orally bioavailable form of propofol. For example, gastric retention oral dosage forms can be appropriate for compounds absorbed primarily from the upper gastrointestinal tract, and sustained release oral dosage forms can be appropriate for compounds absorbed primarily form the lower gastrointestinal tract.

Certain compounds are absorbed primarily from the small intestine. In general, compounds traverse the length of the small intestine in about 3 to 5 hours. For compounds that are not easily absorbed by the small intestine or that do not dissolve readily, the window for active agent absorption in the small intestine may be too short to provide a desired therapeutic effect.

Gastric retention dosage forms, i.e., dosage forms that are designed to be retained in the stomach for a prolonged period of time, can increase the bioavailability of drugs that are most readily absorbed by the upper gastrointestinal tract. The residence time of a conventional dosage form in the stomach is 1 to 3 hours. After transiting the stomach, there is approximately a 3 to 5 hour window of bioavailability before the dosage form reaches the colon. However, if the dosage form is retained in the stomach, the drug can be released before it reaches the small intestine and will enter the intestine in solution in a state in which it can be more readily absorbed. Another use of gastric retention dosage forms is to improve the bioavailability of a drug that is unstable to the basic conditions of the intestine (see, e.g., Hwang et al., Critical Reviews in Therapeutic Drug Carrier Systems, 1998, 15, 243-284).

To enhance drug absorption from the upper gastrointestinal tract, several gastric retention dosage forms have been developed. Examples include, hydrogels (see, e.g., Gutierrez-Rocca et al., U.S. Application Publication No. 2003/0008007), buoyant matrices (see, e.g., Lohray et al., Application Publication No. 2006/0013876), polymer sheets (see, e.g., Mohammad, Application Publication No. 2005/0249798), microcellular foams (see, e.g., Clarke et al., Application Publication No. 2005/0202090), swellable dosage forms (see, e.g., Edgren et al., U.S. Application Publication No. 2005/0019409; Edgren et al., U.S. Pat. No. 6,797,283; Jacob et al., U.S. Application Publication No. 2006/0045865; Ayres, U.S. Application Publication No. 2004/0219186; Gusler et al., U.S. Pat. No. 6,723,340; Flashner-Barak et al., U.S. Pat. No. 6,476,006; Wong et al., U.S. Pat. Nos. 6,120,803; 6,548,083; and Shell et al., U.S. Pat. No. 6,635,280 and U.S. Pat. No. 5,780,057); bioadhesive polymers (see, e.g., Mathiowitz et al., U.S. Pat. No. 6,235,313; U.S. Pat. No. 6,207,197; and Jacob et al., U.S. Application Publication Nos. 2006/0045865 and 2005/0064027); and ion exchange resins.

In a swelling and expanding system, dosage forms that swell and change density in relation to the surrounding gastric content can be retained in the stomach for longer than a conventional dosage form. A dosage form can absorb water and swell to form a gelatinous outside surface and float on the surface of gastric content surface while maintaining integrity before releasing a drug. Fatty materials can be added to impede wetting and enhance flotation when hydration and swelling alone are insufficient. Materials that release gases may also be incorporated to reduce the density of a gastric retention dosage form. Swelling also can significantly increase the size of a dosage form and thereby impede discharge of the non-disintegrated swollen solid dosage form through the pylorus into the small intestine. Swellable dosage forms can be formed by encapsulating a core containing drug and a swelling agent, or by combining a drug, swelling agent, and one or more erodible polymers.

Gastric retention dosage forms can also be in the form of a folded thin sheet containing a drug and water-insoluble diffusible polymer that opens in the stomach to its original size and shape, which is sufficiently large to prevent or inhibit passage of the expanded dosage from through the pyloric sphincter.

Floating and buoyancy gastric retention dosage forms can be designed to trap gases within sealed encapsulated cores that can float on the gastric contents, and thereby be retained in the stomach for a longer time, e.g., 9 to 12 hours. Due to the buoyancy effect, these systems can provide a protective layer preventing the reflux of gastric content into the esophageal region and can also be used for controlled release devices. A floating system can, for example, contain hollow cores containing drug coated with a protective membrane. The trapped air in the cores floats the dosage from on the gastric content until the soluble ingredients are released and the system collapses. In other floating systems, cores contain drug and chemical substances capable of generating gases when activated. For example, coated cores, containing carbonate and/or bicarbonate can generate carbon dioxide in the reaction with hydrochloric acid in the stomach or incorporated organic acid in the system. The gas generated by the reaction is retained to float the dosage form. The inflated dosage form later collapses and clears form the stomach when the generated gas permeates slowly through the protective coating.

Bioadhesive polymers can also provide a vehicle for controlled delivery of drugs to a number of mucosal surfaces in addition to the gastric mucosa (see, e.g., Mathiowitz et al., U.S. Pat. No. 6,235,313; U.S. Pat. No. 6,207,197). A bioadhesive system can be designed by incorporation of a drug and other excipients within a bioadhesive polymer. On ingestion, the polymer hydrates and adheres to the mucus membrane of the gastrointestinal tract. Bioadhesive polymers can be selected that adhere to a desired region or regions of the gastrointestinal tract. Bioadhesive polymers can be selected to optimized delivery to targeted regions of the gastrointestinal tract including the stomach and small intestine. The mechanism of the adhesion is thought to be through the formation of electrostatic and hydrogen bonding at the polymer-mucus boundary. Jacob et al., U.S. Application Publication Nos. 2006/0045865 and 2005/0064027 disclose bioadhesive delivery systems which are useful for drug delivery to both the upper and lower gastrointestinal tract.

Ion exchange resins have been shown to prolong gastric retention, potentially by adhesion.

Gastric retention oral dosage forms can be appropriately used for delivery of drugs that are absorbed mainly from the upper gastrointestinal tract. For example, certain propofol prodrugs such as (S)-2-amino-3-(2,6-diisopropylphenoxycarbonyloxy)-propanoic acid exhibit limited colonic absorption, and are absorbed primarily from the upper gastrointestinal tract. Thus, dosage forms that release (S)-2-amino-3-(2,6-diisopropylphenoxycarbonyloxy)-propanoic acid in the upper gastrointestinal tract and/or retard transit of the dosage form through the upper gastrointestinal tract will tend to enhance the oral bioavailability of the propofol prodrug. Other forms of propofol disclosed herein are absorbed primarily from the upper gastrointestinal tract, such as the small intestine, and therefore can be appropriately used with gastric retention dosage forms.

Polymer matrices have also been used to achieve controlled release of the drug over a prolonged period of time. Such sustained or controlled release can be achieved by limiting the rate by which the surrounding gastric fluid can diffuse through the matrix and reach the drug, dissolve the drug and diffuse out again with the dissolved drug, or by using a matrix that slowly erodes, continuously exposing fresh drug to the surrounding fluid. Disclosures of polymer matrices that function by these methods are found, for example, in Skinner, U.S. Pat. Nos. 6,210,710, 6,217,903; Rencher et al., U.S. Pat. No. 5,451,409; Kim, U.S. Pat. No. 5,945,125; Kim, PCT International Publication No. WO 96/26718; Ayer et al., U.S. Pat. No. 4,915,952; Akhtar et al., U.S. Pat. No. 5,328,942; Fassihi et al., U.S. Pat. No. 5,783,212; Wong et al., U.S. Pat. No. 6,120,803; and Pillay et al., U.S. Pat. No. 6,090,411.

Other drug delivery devices that remain in the stomach for extended periods of time include, for example, hydrogel reservoirs containing particles (U.S. Pat. No. 4,871,548); swellable hydroxypropylmethylcellulose polymers (U.S. Pat. No. 4,871,548); planar bioerodible polymers (U.S. Pat. No. 4,767,627); plurality of compressible retention arms (U.S. Pat. No. 5,443,843); hydrophilic water-swellable, cross-linked polymer particles (U.S. Pat. No. 5,007,790); and albumin-cross-linked polyvinylpyrrolidone hydrogels (Park et al, J Controlled Release 1992, 19, 131-134).

In certain embodiments, pharmaceutical compositions provided by the present disclosure can be practiced with a number of different dosage forms, which can be adapted to provide sustained release of an anti-emetic compound and a highly orally bioavailable form of propofol upon oral administration. Sustained release oral dosage forms can be used to release drugs over a prolonged time period and are useful when it is desired that a drug or drug form be delivered to the lower gastrointestinal tract. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art (see, for example, “Remington's Pharmaceutical Sciences,” Lippincott, Williams & Wilkins, 21st edition, 2005, Chapters 46 and 47; Langer, Science 1990, 249, 1527-1533; and Rosoff, “Controlled Release of Drugs,” 1989, Chapter 2).

Sustained release oral dosage forms include any oral dosage form that maintains therapeutic plasma, blood or tissue levels of a drug for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art (see, for example, “Remington's: The Science and Practice of Pharmacy,” Lippincott, Williams & Wilkins, 21st edition, 2005, Chapters 46 and 47; Langer, Science 1990, 249, 1527-1533; and Rosoff, “Controlled Release of Drugs,” 1989, Chapter 2).

In diffusion-controlled systems, a water-insoluble polymer controls the flow of fluid and the subsequent egress of dissolved drug from the dosage form. Both diffusional and dissolution processes are involved in release of drug from the dosage form. In reservoir devices, a core comprising a drug is coated with the polymer, and in matrix systems, the drug is dispersed throughout the matrix. Cellulose polymers such as ethylcellulose or cellulose acetate can be used in reservoir devices. Examples of materials useful in matrix systems include methacrylates, acrylates, polyethylene, acrylic acid copolymers, polyvinylchloride, high molecular weight polyvinylalcohols, cellulose derivates, and fatty compounds such as fatty acids, glycerides, and carnauba wax.

In dissolution-controlled systems, the rate of dissolution of the drug is controlled by slowly soluble polymers or by microencapsulation. Once the coating is dissolved, the drug becomes available for dissolution. By varying the thickness and/or the composition of the coating or coatings, the rate of drug release can be controlled. In some dissolution-controlled systems, a fraction of the total dose can comprise an immediate-release component. Dissolution-controlled systems include encapsulated/reservoir dissolution systems and matrix dissolution systems. Encapsulated dissolution systems can be prepared by coating particles or granules of drug with slowly soluble polymers of different thickness or by microencapsulation. Examples of coating materials useful in dissolution-controlled systems include gelatin, carnauba wax, shellac, cellulose acetate phthalate, and cellulose acetate butyrate. Matrix dissolution devices can be prepared, for example, by compressing a drug with a slowly soluble polymer carrier into a tablet form.

The rate of release of drug from osmotic pump systems is determined by the inflow of fluid across a semipermeable membrane into a reservoir, which contains an osmotic agent. The drug is either mixed with the agent or is located in a reservoir. The dosage form contains one or more small orifices from which dissolved drug is pumped at a rate determined by the rate of entrance of water due to osmotic pressure. As osmotic pressure within the dosage form increases, the drug is released through the orifice(s). The rate of release is constant and can be controlled within tight limits yielding relatively constant plasma and/or blood concentrations of the drug. Osmotic pump systems can provide a constant release of drug independent of the environment of the gastrointestinal tract. The rate of drug release can be modified by altering the osmotic agent and the sizes of the one or more orifices.

The release of drug from erosion-controlled systems is determined by the erosion rate of a carrier matrix. Drug is dispersed throughout the polymer and the rate of drug release depends on the erosion rate of the polymer. The drug-containing polymer can degrade from the bulk and/or from the surface of the dosage form.

Sustained release oral dosage forms can be in any appropriate form for oral administration, such as, for example, in the form of tablets, pills, or granules. Granules can be filled into capsules, compressed into tablets, or included in a liquid suspension. Sustained release oral dosage forms can additionally include an exterior coating to provide, for example, acid protection, ease of swallowing, flavor, identification, and the like.

In certain embodiments, sustained release oral dosage forms can comprise a therapeutically effective amount of an anti-emetic compound, a highly orally bioavailable form of propofol, and a pharmaceutically acceptable vehicle. In certain embodiments, sustained release oral dosage forms can comprise less than a therapeutically effective amount of an anti-emetic compound and a highly orally bioavailable form of propofol, and a pharmaceutically effective vehicle. Multiple sustained release oral dosage forms, each dosage form comprising less than a therapeutically effective amount of an anti-emetic compound and a highly orally bioavailable form of propofol, may be administered at a single time or over a period of time to provide a therapeutically effective dose or regimen for treating emesis in a patient.

Sustained release oral dosage forms provided by the present disclosure may release an anti-emetic compound and a highly orally bioavailable form of propofol from the dosage form to facilitate the ability of the anti-emetic compound and the highly orally bioavailable form of propofol to be absorbed from an appropriate region of the gastrointestinal tract, for example, in the small intestine, or in the colon. In certain embodiments, a sustained release oral dosage form can release an anti-emetic compound and a highly orally bioavailable form of propofol from the dosage form over a period of at least about 4 hours, at least about 8 hours, at least about 12 hours, at least about 16 hours, at least about 20 hours, and in certain embodiments, at least about 24 hours. In certain embodiments, a sustained release oral dosage form can release an anti-emetic compound and a highly orally bioavailable form of propofol from the dosage form in a delivery pattern of from about 0 wt % to about 20 wt % in about 0 to about 4 hours, about 20 wt % to about 50 wt % in about 0 to about 8 hours, about 55 wt % to about 85 wt % in about 0 to about 14 hours, and about 80 wt % to about 100 wt % in about 0 to about 24 hours. In certain embodiments, a sustained release oral dosage form can release an anti-emetic compound and a highly orally bioavailable form of propofol from the dosage form in a delivery pattern of from about 0 wt % to about 20 wt % in about 0 to about 4 hours, about 20 wt % to about 50 wt % in about 0 to about 8 hours, about 55 wt % to about 85 wt % in about 0 to about 14 hours, and about 80 wt % to about 100 wt % in about 0 to about 20 hours. In certain embodiments, a sustained release oral dosage form can release an anti-emetic compound and a highly orally bioavailable form of propofol from the dosage form in a delivery pattern of from about 0 wt % to about 20 wt % in about 0 to about 2 hours, about 20 wt % to about 50 wt % in about 0 to about 4 hours, about 55 wt % to about 85 wt % in about 0 to about 7 hours, and about 80 wt % to about 100 wt % in about 0 to about 8 hours.

Sustained release oral dosage forms comprising a highly orally bioavailable form of propofol can provide a concentration of propofol in the plasma, blood, or tissue of a patient over time, following oral administration to the patient. The concentration profile of propofol can exhibit an AUC that is proportional to the dose of the corresponding highly orally bioavailable form of propofol.

Regardless of the specific controlled release oral dosage form used, the anti-emetic compound and the highly orally bioavailable form of propofol can be released from an orally administered dosage form over a sufficient period of time to provide prolonged therapeutic concentrations of the anti-emetic compound and propofol in the plasma and/or blood of a patient. Following oral administration, a dosage form comprising an anti-emetic compound and a highly orally bioavailable form of propofol can provide a therapeutically effective concentration of the anti-emetic compound and a therapeutically effective concentration of propofol in the plasma and/or blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous time periods during which a therapeutically effective concentration of the anti-emetic compound and propofol is maintained can be the same or different. Also, to be therapeutically effective, the plasma or blood concentration profile of an anti-emetic compound and the plasma or blood concentration profile of propofol following oral administration can be, although need not be the same. The continuous period of time during which a therapeutically effective plasma concentration of the anti-emetic compound and propofol is maintained can begin shortly after oral administration or after a time interval. A therapeutically effect plasma concentration of an anti-emetic compound can begin at the same or a different time than that of propofol, e.g., the therapeutically effective plasma concentration window for an anti-emetic compound may or may not begin at a different time than that of propofol, may or may not overlap, and/or may or may not have the same duration.

In certain embodiments, an oral dosage for treating emesis in a patient comprises an anti-emetic compound and a highly orally bioavailable form of propofol, wherein the oral dosage form is adapted to provide, after a single administration of the oral dosage form to the patient, a therapeutically effective concentration of the anti-emetic compound in the plasma of the patient for a first continuous time period selected from at least about 4 hours, at least about 8 hours, at least about 12 hours, and at least about 16 hours, and at least about 20 hours, and a therapeutically effective concentration of propofol in the plasma of the patient for a second continuous time period independently selected from at least about 4 hours, at least about 8 hours, at least about 12 hours, at least about 16 hours, and at least about 20 hours. The continuous time periods during which a therapeutically effective concentration of the anti-emetic compound and propofol is maintained can be the same or different.

In certain embodiments, it can be desirable that the plasma and/or blood concentration of propofol be maintained at a level between a concentration that causes sedation in the patient and a minimum therapeutically effective concentration for treating emesis for a continuous period of time. The plasma concentration of propofol that causes sedation or anesthesia in a patient can vary depending on the individual patient. It is estimated that a plasma propofol concentration from about 1,500 ng/mL to about 2,000 ng/mL will produce sedation, while a plasma propofol concentration from about 3,000 ng/mL to about 10,000 ng/mL is sufficient to maintain general anesthesia. In certain embodiments, a minimum therapeutically effective plasma propofol concentration can be 10 ng/mL, 20 ng/mL, 50 ng/mL, 100 ng/mL, 100 ng/mL, 200 ng/mL, 400 ng/mL, or 600 ng/mL. In certain embodiments, a therapeutically effective plasma concentration of propofol for treating emesis is from about 10 ng/mL to less than a sedative concentration. In certain embodiments, a therapeutically effective plasma concentration of propofol for treating emesis is from about 200 ng/mL to about 1,000 ng/mL. In certain embodiments, a dosage form can provide a plasma propofol concentration that, following oral administration to a patient, does not produce sedation and/or anesthesia in the patient.

A therapeutically effective propofol plasma concentration for treating emesis in a patient can also be defined in terms of the plasma concentration profile. Thus, in certain embodiments, following oral administration of a dosage form to a patient, the maximum plasma propofol concentration, Cₘₐₓ, is less than that which causes sedation, for example, is less than about 1,500 ng/mL to about 2,000 ng/mL. In certain embodiments, following oral administration of a dosage form to a patient, the plasma propofol AUC during a 4-hour period can range from about 1,000 ng·h/mL to about 3,200 ng·h/mL and not cause sedation at any time following oral administration.

In certain embodiments, following oral administration of a dosage form to a patient, the plasma propofol AUC during an 8-hour period can range from about 1,600 ng·h/mL to about 6,400 ng·h/mL and not cause sedation at any time following oral administration.

In certain embodiments, following oral administration of a dosage form to a patient, the plasma propofol AUC during a 12-hour period can range from about 2,400 ng·h/mL to about 9,200 ng·h/mL and not cause sedation at any time following oral administration.

In certain embodiments, following oral administration of a dosage form to a patient, the plasma propofol AUC during a 16-hour period can range from about 3,200 ng·h/mL to about 12,800 ng·h/mL and not cause sedation at any time following oral administration.

In certain embodiments, following oral administration of a dosage form to a patient, the plasma propofol AUC during a 32-hour period can range from about 4,000 ng·h/mL to about 16,000 ng·h/mL and not cause sedation at any time following oral administration.

### Methods of Treating Emesis

Methods provided by the present disclosure may be used to treat emesis of any etiology. Emesis may be induced by factors including, but not limited to, cancer chemotherapeutic agents such as alkylating agents, e.g., cyclophosphamide, carmustine, lomustine, and chlorambucil; cytotoxic antibiotics, e.g., dactinomycin, doxorubicin, mitomycin-C, and bleomycin; anti-metabolites, e.g., cytarabine, methotrexate, and 5-fluorouracil; vinca alkaloids, e.g., etoposide, vinblastine, and vincristine; and other chemotherapeutic agents such as cisplatin, dacarbazine, procarbazine, and hydroxyurea; and combinations thereof; radiation sickness; radiation therapy, e.g., irradiation of the thorax or abdomen, such as in the treatment of cancer; poisons; toxins such as toxins caused by metabolic disorders or by infection, e.g., gastritis, or released during bacterial or viral gastrointestinal infection; pregnancy; vestibular disorders, such as motion sickness, vertigo, dizziness, and Meniere's disease; post-operative sickness; gastrointestinal obstruction; reduced gastrointestinal motility; visceral pain, such as myocardial infarction or peritonitis; headache; migraine; increased intracranial pressure; decreased intracranial pressure (e.g., altitude sickness); opioid analgesics such as morphine; drugs that cause gastric irritation such as nonsteroidal anti-inflammatory drugs, selective serotonin reuptake inhibitors, antibiotics, and antiparasitics; drugs that indirectly stimulate the vomiting center such as morphine, digitoxin, alcohol, ipecac, and chemotherapy drugs; olfactory, visual, vestibular, and psychogenic stimuli; anesthetics; pancreatitis; diabetic ketoacidosis; meningitis; heart failure; hepatobiliary causes; cerebrovascular trauma; hypotension; peridonitis; hyponatremia; brain tumors; myocardial infarction; gastrointestinal bleeding; uremia; hypercalcemia; gastroesophageal reflux disease; acid indigestion; over-indulgence of food or drink; acid stomach; sour stomach; regurgitation; heartburn such as episodic heartburn, nocturnal heartburn and meal-induced heartburn; and dyspepsia.

Emesis can also be caused by conditions, disorders, or diseases of the gastrointestinal tract such as cholecystitis, choledocholithiasis, intestinal obstruction, acute gastroenteritis, perforated viscus, dyspepsia resulting from, for example, gastroesophageal reflux disease, peptic ulcer disease, gastroparesis, gastric or esophageal neoplasms, infiltrative gastric disorders such as Menetrier's syndrome, Crohn's disease, eosinophilic gastroenteritis, sarcoidosis and amuloidosis, gastric infections such as CMV, fingal, TB, and syphilis, parasites such as Giardia lamblia and Strongyloides stercoralis, chronic gastric volvulus, chronic intestinal ischemia, altered gastric motility and/or food intolerance, or Zollinger-Ellison syndrome.

In certain embodiments, pharmaceutical compositions and dosage forms provided by the present disclosure may be used to treat acute emesis induced by chemotherapy or by radiation associated with cancer therapy. In certain embodiments, pharmaceutical compositions and dosage forms provided by the present may be used to treat delayed emesis induced by chemotherapy or by radiation associated with cancer therapy. In certain embodiments, pharmaceutical compositions and dosage forms provided by the present disclosure may be used to treat anticipatory emesis induced by chemotherapy or by radiation associated with cancer therapy.

In certain embodiments, methods provided by the present disclosure may be used to treat CINV, PONV, or emesis induced by radiation therapy. In certain embodiments, for treating CINV in a patient, a form of propofol and an anti-emetic compound useful for treating CINV such as aprepitant, dexamethasone, dolasetron, dronabinol, granisetron, lorazepam, metoclopramide, ondonsetron, palonosetrondiphenhydramine, prochlorperazine, or a combination of any of the foregoing, may be orally administered to a patient in need of such treatment. In certain embodiments, methods provided by the present disclosure may be used to treat emesis induced by a cancer chemotherapeutic agent, radiation, anesthesia, a poison, a toxin, pregnancy, a vestibular disorder, surgery, gastrointestinal obstruction, reduced gastrointestinal motility, visceral pain, migraine, increased intracranial pressure, decreased intracranial pressure, or an opioid analgesic. In certain embodiments, methods provided by the present disclosure may be used to treat emesis induced by chemotherapy or radiation associated with cancer therapy.

In certain embodiments, methods of treating emesis in a patient comprise orally administering to a patient in need of such treatment a therapeutically effective amount of a first anti-emetic compound selected from a serotonin 5-HT₃ receptor antagonist, a histamine receptor antagonist, a dopamine receptor antagonist, a muscarinic receptor antagonist, an acetylcholine receptor antagonist, a cannabinoid receptor antagonist, a limbic system inhibitor, a NK-1 receptor antagonist, a corticosteroid, a tachykinin antagonist, a GABA agonist, a substance P inhibitor, and combinations of any of the foregoing, and an oral dosage form comprising a highly orally bioavailable form of propofol, wherein the oral dosage form is adapted to provide, after a single oral administration of the oral dosage form to the patient, a therapeutically effective concentration of propofol in the plasma of the patient during a continuous time period independently selected from at least about 4 hours, at least about 8 hours, at least about 12 hours, at least about 16 hours, and at least about 20 hours.

In certain embodiments, for treating PONV in a patient, a highly orally bioavailable form of propofol and an anti-emetic compound useful for treating PONV such as dexamethasone, dolasetron, granisetron, metoclopramide, ondansetron, tropisetron, droperidol, dimenhydrinate, ephedrine, prochlorperazine, promethazine, or a combination of any of the foregoing, may be orally administered to a patient in need of such treatment.

In certain embodiments, for treating emesis induced by radiotherapy in a patient, a highly orally bioavailable form of propofol and an anti-emetic compound useful for treating emesis induced by radiotherapy such as granisetron, ondansetron, dexamethasone, or a combination of any of the foregoing, may be orally administered to a patient in need of such treatment.

When treating breakthrough emesis in a patient a highly orally bioavailable form of propofol and an anti-emetic compound useful for treating breakthrough emesis such as prochlorperazine, thiethylperazine, metoclopramide, diphenhydramine, lorzepam, haloperidol, dronabinol, ondansetron, granisetron, dolasetron, dexamethasone, olanzapine, promethazine, or a combination of any of the foregoing, may be orally administered to a patient in need of such treatment.

In certain embodiments, for treating anticipatory emesis, a highly orally bioavailable form of propofol and an anti-emetic compound useful for treating anticipatory emesis such as alprazolam and lorazepam, or a combination of any of the foregoing, may be orally administered to a patient in need of such treatment.

Methods provided by the present disclosure include treating acute emesis, delayed emesis, anticipatory emesis, breakthrough emesis, refractory emesis, and chronic emesis associated with the treatment of cancer including chemotherapy and radiation therapy. Acute emesis occurs within the first 24 hours after the administration of chemotherapy, usually within the first 1 to 2 hours. Acute emesis type is believed to be initiated by stimulation primarily of dopamine and serotonin receptors in the CTZ, which triggers the vomiting cascade. Delayed emesis begins at least 24 hours after the administration of chemotherapy and may last up to 120 hours. Patients who experience acute CINV are more likely to also experience delayed emesis. The causative mechanism in delayed emesis is not well defined, but the metabolites of the administered chemotherapeutic agents are thought to continue to affect the central nervous system and the gastrointestinal tract. Anticipatory emesis occurs as a result of an unpleasant experience with chemotherapy. It occurs as the person is preparing for the next dose of chemotherapy where the person anticipates that emesis will occur as it did before. Anticipatory emesis occurs before the beginning of a new cycle of chemotherapy, in response to conditioned stimuli such as the smells, sights, and sounds of the treatment room, or the presence of a specific person designated to administer the chemotherapy. Anticipatory emesis usually occurs 12 hours before administration of chemotherapy in patients who have experienced failed control of emesis in previous treatments. Breakthrough emesis occurs despite preventive therapy and requires additional therapy. Anti-emetic treatment administered to patients who have not responded to prophylactic regimens is often referred to as rescue therapy. Refractory emesis occurs after one, a few or several chemotherapy treatments even though the person is being treated to prevent or control emesis. The anti-emesis is characterized by the presence of continuous or intermittent symptoms of emesis for more than about one week.

Pharmaceutical compositions provided by the present disclosure and dosage forms provided by the present disclosure comprising a pharmaceutical composition may be administered prior to the commencement of and/or after an event anticipated to induce emesis. In certain embodiments, administration may commence from about 0 minutes to about 10 hours prior to such an event, from about 0 minutes to about 5 hours prior to such an event, and in certain embodiments, from about 0 minutes to about 3 hours prior to the event. Following an event anticipated to induce emesis, administration may be at time intervals and for a duration sufficient to prevent or ameliorate emesis. The frequency and course of administration may be a prescribed regimen of treatment. Administration may also be on an as-needed basis, for example, a pharmaceutical composition provided by the present disclosure may be administered when a patient experiences anticipatory symptoms of emesis, or after a patient manifests emesis. Pharmaceutical compositions provided by the present disclosure, being adapted for oral administration of both an anti-emetic compound and a highly orally bioavailable form of propofol, may be particularly useful in treating delayed emesis, breakthrough emesis, chronic emesis, or other type of emesis that does not necessarily manifest in a clinical setting.

In certain embodiments, methods provided by the present disclosure provide for the oral coadministration of an anti-emetic compound and a highly orally bioavailable form of propofol to treat emesis. Co-administration includes administering the anti-emetic compound and the highly orally bioavailable form of propofol simultaneously, such as in a single pharmaceutical composition or dosage form, for example, a capsule or tablet having a fixed ratio of the first and second amounts, or in multiple, separate capsules or tablets. When an anti-emetic compound and a highly orally bioavailable form of propofol are co-administered in multiple separate dosage forms, each dosage form can contain both the anti-emetic compound and the highly orally bioavailable form of propofol in amounts less than a therapeutically effective amount, and thus more than one dosage form is administered to provide a therapeutically effective dose. In certain embodiments, an anti-emetic compound and a highly orally bioavailable form of propofol are contained in separate dosage forms, and orally co-administered. Coadministration also includes orally administering the anti-emetic compound and the highly orally bioavailable form of propofol separately in a sequential manner, in either order. When coadministration involves the separate administration of the anti-emetic compound and the highly orally bioavailable form of propofol, the compound and highly orally bioavailable form of propofol are administered at a time interval to provide a desired therapeutic effect.

In certain embodiments, co-administration may also comprise both simultaneous and sequential administration. For example, at the inception of a treatment regimen, an anti-emetic compound and a highly orally bioavailable form of propofol may be administered simultaneously. Then at a later time during the treatment regimen either the anti-emetic compound, the highly orally bioavailable form of propofol, or both the anti-emetic compound and the highly orally bioavailable form of propofol can be administered.

In certain embodiments, an anti-emetic compound and a highly orally bioavailable form of propofol may be administered prior to an event anticipated to induce emesis, for example, 30 minutes to 2 hours prior to the inception of chemotherapy to treat CINV, prior to the administration of anesthesia to treat PONV, or prior to radiotherapy. Following the initial administration, a first anti-emetic compound, a highly orally bioavailable form of propofol, or a combination of an anti-emetic compound and highly orally bioavailable form of propofol may be administered to the patient at intervals of minutes, hours, or days. Administration of the anti-emetic compound, highly orally bioavailable form of propofol, or both may continue as necessary to effectively treat emesis in the patient. The dosage of an anti-emetic compound and/or the dosage of a highly orally bioavailable form of propofol may be the same or different at each administration. For example, a dosage of an anti-emetic compound and a highly orally bioavailable form of propofol may be administered initially. In subsequent doses, the dosage of the first anti-emetic compound may be reduced and the dosage of the highly orally bioavailable form of propofol may remain the same or increase. In certain embodiments, subsequent doses may comprise none of the anti-emetic compound and only a therapeutically effective amount of a highly orally bioavailable form of propofol.

Thus, methods provided by the present disclosure include treating emesis in a patient comprising simultaneously administering a first anti-emetic compound and a highly orally bioavailable form of propofol, and administering a first anti-emetic compound and a highly orally bioavailable form of propofol during the course of a regimen in which the first anti-emetic compound and the highly orally bioavailable form of propofol are administered simultaneously and/or are not administered simultaneously.

In certain embodiments, a highly orally bioavailable form of propofol may be co-administered with an anti-emetic compound that is effective in treating acute emesis. For example, certain 5-HT₃ receptor antagonists such as ondansetron are considered to be more effective in treating acute emesis relative to delayed emesis induced by chemotherapeutic agents. 5-HT₃ receptor antagonist are well-tolerated agents that are widely used in the U.S. to prevent acute emesis associated with chemotherapy or radiation therapy, but studies have failed to support their benefit in the management of delayed emesis (Gandara et al., Semin Oncol. 1992, 19, 67-71). Furthermore, although the serotonin 5-HT₃ receptor antagonists represent a major improvement in the management of chemotherapy-induced emesis, clinical experience indicates that the anti-emetic efficacy of serotonin 5-HT₃ receptor antagonists (given as single agents or in combination with dexamethasone) is not always maintained over multiple chemotherapy cycles (Morrow et al., Support Care Cancer 1998, 6, 46-50; de Wit et al., Brit J. Cancer 1998, 77, 1487-1491).

A therapeutically effective amount of an anti-emetic compound and a highly orally bioavailable form of propofol will depend on a number of factors, including, but not limited to, the age, sex and weight of the patient, the current medical condition of the patient, the etiology of the emesis, the severity of the emesis, past experience with emesis, past experience with anti-emetic compounds, past experience with highly orally bioavailable forms of propofol, and the like. A therapeutically effective amount can also depend on the potency of an anti-emetic compound, the potency of a highly orally bioavailable form of propofol, any synergistic effects, and the potential side effects of an anti-emetic compound and/or a highly orally bioavailable form of propofol. A therapeutically effective amount will also depend on the judgment of the prescribing physician.

An appropriate dosage of an anti-emetic compound and/or highly orally bioavailable form of propofol may be determined according to any one of several well-established protocols. For example, animal studies, such as studies using mice or rats, may be used to determine an appropriate dose of a pharmaceutical compound. The results from animal studies can be extrapolated to determine doses for use in other species, such as for example, humans.

In certain embodiments, a dose may comprise a therapeutically effective amount of a first anti-emetic compound and a therapeutically effective amount of a highly orally bioavailable form of propofol. In certain embodiments, a dose may comprise and amount of a first anti-emetic compound and an amount of a highly orally bioavailable form of propofol, each amount being less than a therapeutically effective amount, but which amounts together comprise a therapeutically effective amount. Accordingly, methods provided by the present disclosure include amounts of a first anti-emetic compound and a highly orally bioavailable form of propofol that act independently to effect treatment of emesis. Furthermore, methods provided by the present disclosure include an amount of a first anti-emetic compound and an amount of a highly orally bioavailable form of propofol that act additively and/or synergistically to effect treatment of emesis. Whether the anti-emetic compound and the highly orally bioavailable form of propofol act independently or additively/synergistically to effect treatment of emesis may depend on, for example, the etiology of the emesis, the time after which the emesis was induced, the severity of the emesis, the condition of the patient, the patient's past experience with anti-emesis treatment, and the like.

In certain embodiments, dosage forms provided by the present disclosure are adapted to be administered to a patient no more than twice per day, and in certain embodiments, only once per day, for example, twice per day, three times per day, or four times per day. Dosing may be provided alone or in combination with other drugs or therapy for treating emesis or disease or condition other than emesis and may continue as long as required for effective treatment of the emesis.

Pharmaceutical compositions and oral dosage forms provided by the present disclosure can include, in addition to an anti-emetic compound and a highly orally bioavailable form of propofol, one or more additional therapeutic agents effective for treating emesis or a different disease, disorder, or condition. Methods by the present disclosure include orally administering one or more compounds or pharmaceutical compositions provided by the present disclosure and one or more other therapeutic agents provided that the combined administration does not inhibit the therapeutic efficacy of the anti-emetic compound and the highly orally bioavailable form of propofol and/or does not produce adverse combination effects.

In certain embodiments, pharmaceutical compositions and oral dosage forms provided by the present disclosure may be administered concurrently with the administration of another therapeutic agent, which may be part of the same pharmaceutical composition as, or in a different pharmaceutical composition. In certain embodiments, compounds provided by the present disclosure may be administered prior or subsequent to administration of another therapeutic agent. In certain embodiments of combination therapy, the combination therapy can comprise alternating between administering a composition provided by the present disclosure and a composition comprising another therapeutic agent, e.g., to minimize adverse side effects associated with a particular drug. When a pharmaceutical composition provided by the present disclosure is administered concurrently with another therapeutic agent that potentially can produce adverse side effects including, but not limited to, toxicity, the other therapeutic agent may advantageously be administered at a dose that falls below the threshold at which the adverse side effect is elicited.

Pharmaceutical compositions, dosage forms, and methods provided by the present disclosure may be evaluated for efficacy in treating emesis using any suitable animal model or based on clinical trials. For example, efficacy in treating emesis induced by chemotherapeutic agents can be determined based on effects indicative of emesis such as pica, gastric stasis, and reduced food intake in rats, mice, or ferrets (see, e.g., Liu et al., Physiology & Behavior, 2005, 85, 271-277; Endo et al., Biogenic Amines, 2004, 18(3-6), 419-434; and Malik et al., Eur. J. Pharmacol, 2007, 555, 164-173. In clinical trials, assessment instruments such as the Duke Descriptive Scale, Visual Analog Scales, Morrow Assessment of Nausea and Emesis, Rhodes Index of Nausea and Vomiting Form-2, and Functional Living Index Emesis can be used to measure efficacy (see, e.g., Rhodes et al., CA Cancer J Clin, 2001, 51, 232-248 and references therein). In general, adequately controlled, double blind placebo controlled trails may be used to evaluate efficacy in humans.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

What is claimed is:

1. A pharmaceutical composition comprising:
a first anti-emetic compound selected from a serotonin 5-HT₃ receptor antagonist, a histamine receptor antagonist, a dopamine receptor antagonist, a muscarinic receptor antagonist, an acetylcholine receptor antagonist, a cannabinoid receptor antagonist, a limbic system inhibitor, a NK-1 receptor antagonist, a corticosteroid, a tachykinin antagonist, a GABA agonist, a substance P inhibitor, and combinations of any of the foregoing; and
a highly orally bioavailable form of propofol that exhibits an oral bioavailability that is at least 10 times greater than the oral bioavailability of propofol when orally administered in an equivalent dosage form.

2. The pharmaceutical composition of claim 1, wherein the highly orally bioavailable form of propofol is selected from a propofol prodrug and a propofol tight-ion pair complex.

3. The pharmaceutical composition of claim 2, wherein the highly orally bioavailable form of propofol is a propofol prodrug and is selected from a compound of Formula (I) to Formula (XIII), a pharmaceutically acceptable salt of any of the foregoing, a pharmaceutically acceptable solvate of any of the foregoing, and a combination of any of the foregoing.

4. The pharmaceutical composition of claim 3, wherein the propofol prodrug is (S)-2-amino-3-(2,6-diisopropylphenoxycarbonyloxy)-propanoic acid, a pharmaceutically acceptable salt thereof, or a pharmaceutically acceptable solvate of any of the foregoing.

5. The pharmaceutical composition of claim 1, wherein the first anti-emetic compound is a serotonin 5-HT₃ receptor antagonist and is selected from alosetron, azasetron, bemesetron, cilansetron, dolasetron, granisetron, indisetron, itasetron, ondansetron, palonosetron, ramosetron, tropisetron, and zatosetron.

6. The pharmaceutical composition of claim 1, further comprising a second anti-emetic compound selected from a serotonin 5-HT₃ receptor antagonist, a histamine receptor antagonist, a dopamine receptor antagonist, a muscarinic receptor antagonist, an acetyl choline receptor antagonist, a cannabinoid receptor antagonist, a limbic system inhibitor, a NK-1 receptor antagonist, a corticosteroid, a tachykinin antagonist, a GABA agonist, and a substance P inhibitor.

7. The pharmaceutical composition of claim 6, wherein the second anti-emetic compound is a corticosteroid and is selected from dexamethasone and methylprednisolone.

8. The pharmaceutical composition of claim 7, wherein the first anti-emetic compound is a serotonin 5-HT₃ receptor antagonist, and the highly orally bioavailable form of propofol is (S)-2-amino-3-(2,6-diisopropylphenoxycarbonyloxy)-propanoic acid, a pharmaceutically acceptable salt thereof, or a pharmaceutically acceptable solvate of any of the foregoing.

9. The pharmaceutical composition of claim 1, in an oral dosage form.

10. The pharmaceutical composition of claim 9, wherein the oral dosage form comprises a controlled delivery oral dosage form.

11. The pharmaceutical composition of claim 10, wherein the controlled delivery oral dosage form facilitates absorption of the highly orally bioavailable form of propofol primarily from the small intestine, primarily from the large intestine, or from both the small and large intestine.

12. An oral dosage form for treating emesis in a patient comprising:
a first anti-emetic compound selected from a serotonin 5-HT₃ receptor antagonist, a histamine receptor antagonist, a dopamine receptor antagonist, a muscarinic receptor antagonist, an acetylcholine receptor antagonist, a cannabinoid receptor antagonist, a limbic system inhibitor, a NK-1 receptor antagonist, a corticosteroid, a tachykinin antagonist, a GABA agonist, a substance P inhibitor, and combinations of any of the foregoing; and
a highly orally bioavailable form of propofol, wherein the highly orally bioavailable form of propofol exhibits an oral bioavailability that is at least 5 times greater than the oral bioavailability of propofol when orally administered in an equivalent dosage form;
wherein the oral dosage form is adapted to provide, after a single oral administration of the oral dosage form to the patient:
therapeutically effective concentration of the first anti-emetic compound in the plasma of the patient during a continuous time period selected from at least about 4 hours, at least about 8 hours, at least about 12 hours, and at least about 16 hours, and at least about 20 hours; and
therapeutically effective concentration of propofol in the plasma of the patient during a continuous time period independently selected from at least about 4 hours, at least about 8 hours, at least about 12 hours, at least about 16 hours, and at least about 20 hours.

13. The oral dosage form of claim 12, wherein the concentration of propofol is maintained below a level that causes sedation of the patient.

14. The oral dosage form of claim 12, wherein the therapeutically effective concentration of propofol in the plasma of the patient is from about 10 ng/mL to less than a sedative concentration.

15. The oral dosage form of claim 12, wherein the therapeutically effective concentration of propofol in the plasma of the patient is from about 200 ng/mL to about 1,000 ng/mL.

16. The oral dosage form of claim 12, wherein the first-anti-emetic compound is ondansetron and the therapeutically effective concentration of ondansetron in the plasma of the patient is from about 5 ng/mL to about 50 ng/mL.

17. The oral dosage form of claim 12, wherein the highly orally bioavailable form of propofol is a propofol prodrug and is selected from a compound of Formula (I) to Formula (XIII), a pharmaceutically acceptable salt of any of the foregoing, a pharmaceutically acceptable solvate of any of the foregoing, and a combination of any of the foregoing.

18. The oral dosage form of claim 12, further comprising a second anti-emetic compound selected from a serotonin 5-HT₃ receptor antagonist, a histamine receptor antagonist, a dopamine receptor antagonist, a muscarinic receptor antagonist, an acetyl choline receptor antagonist, a cannabinoid receptor antagonist, a limbic system inhibitor, a NK-1 receptor antagonist, a corticosteroid, a tachykinin antagonist, a GABA agonist, and a substance P inhibitor.

19. The oral dosage form of claim 18, wherein the second anti-emetic compound is a corticosteroid and is selected from dexamethasone and methylprednisolone.

20. A method of treating emesis in a patient comprising administering to a patient in need of such treatment a therapeutically effective amount of the pharmaceutical composition of claim 1.

21. A method of treating emesis in a patient comprising administering to a patient in need of such treatment a therapeutically effective amount of the oral dosage form of claim 12.

22. A method of treating emesis in a patient comprising orally administering to a patient in need of such treatment a therapeutically effective amount of:
a first anti-emetic compound selected from a serotonin 5-HT₃ receptor antagonist, a histamine receptor antagonist, a dopamine receptor antagonist, a muscarinic receptor antagonist, an acetylcholine receptor antagonist, a cannabinoid receptor antagonist, a limbic system inhibitor, a NK-1 receptor antagonist, a corticosteroid, a tachykinin antagonist, a GABA agonist, a substance P inhibitor, and combinations of any of the foregoing; and
an oral dosage form comprising a highly orally bioavailable form of propofol that exhibits an oral bioavailability that is at least 5 times greater than the oral bioavailability of propofol when orally administered in an equivalent dosage form, wherein the oral dosage form is adapted to provide, after a single oral administration of the oral dosage form to the patient a therapeutically effective concentration of propofol in the plasma of the patient during a continuous time period independently selected from at least about 4 hours, at least about 8 hours, at least about 12 hours, at least about 16 hours, and at least about 20 hours.

23. The method of claim 22, wherein the highly orally bioavailable form of propofol is a propofol prodrug, wherein the propofol prodrug exhibits an oral bioavailability at least 5 times greater than the oral bioavailability of propofol when administered in an equivalent dosage form.

24. The method of claim 23, wherein the propofol prodrug is selected from a compound of Formula (I) to Formula (XIII), a pharmaceutically acceptable salt of any of the foregoing, a pharmaceutically acceptable solvate of any of the foregoing, or a combination of any of the foregoing.

25. The method of claim 22, wherein the first anti-emetic compound is a serotonin 5-HT₃ receptor antagonist and is selected from alosetron, azasetron, bemesetron, cilansetron, dolasetron, granisetron, indisetron, itasetron, ondansetron, palonosetron, ramosetron, tropisetron, and zatosetron.

26. The method of claim 22, further comprising orally administering a second anti-emetic compound selected from a serotonin 5-HT₃ receptor antagonist, a histamine receptor antagonist, a dopamine receptor antagonist, a muscarinic receptor antagonist, an acetyl choline receptor antagonist, a cannabinoid receptor antagonist, a limbic system inhibitor, a NK-1 receptor antagonist, a corticosteroid, a tachykinin antagonist, a GABA agonist, and a substance P inhibitor.

27. The method of claim 26, wherein the second anti-emetic compound is a corticosteroid and is selected from dexamethasone and methylprednisolone.
